(19) United States
(12) Patent Application Publication
(10) Pub. No.: US 2011/0020830 A1
(43) Pub. Date: Jan. 27, 2011

(54) DESIGN FOR RAPIDLY CLONING ONE OR MORE POLYPEPTIDE CHAINS INTO AN EXPRESSION SYSTEM

(76) Inventors: Jane C. Schneider, San Diego, CA (US); Charles H. Squires, Poway, CA (US); Huizhu Liu, San Diego, CA (US)

Correspondence Address: WILSON, SONSINI, GOODRICH & ROSATI, 650 PAGE MILL ROAD, PALO ALTO, CA, 94304-1050, US

(21) Appl. No.: 12/933,660

(22) PCT Filed: Mar. 31, 2009
(86) PCT No.: PCT/US09/38895

§ 371 Date: Sep. 20, 2010

Related U.S. Application Data
(60) Provisional application No. 61/041,029, filed on Mar. 31, 2008.

(51) Int. Cl. C12Q 1/68; C12N 15/63

(52) U.S. Cl. 435/6; 435/320.1

(57) ABSTRACT

The present invention provides methods and compositions for the generation and identification of expression constructs that can be used to express sufficient levels of a polypeptide of interest. The compositions include a population of expression vectors, wherein members of the population have a type IIS restriction enzyme recognition site adjacent to a regulatory sequence, and wherein the regulatory element is distinct in at least two members of the population of expression vectors. In various embodiments, the expression vectors further comprise a polynucleotide sequence encoding a polypeptide of interest, wherein the polynucleotide encoding the polypeptide, the polynucleotide of the regulatory sequence, or both, are distinct in at least two members of the population. The compositions are useful for identifying a combination of coding sequences and/or regulatory elements useful for the heterologous expression of the polypeptide of interest.

### FIELD OF THE INVENTION

The present invention relates to molecular biology, particularly to methods and compositions that find utility in the seamless cloning or subcloning of polynucleotides.

### BACKGROUND OF THE INVENTION

More than 150 recombinantly produced proteins and polypeptides have been approved by the U.S. Food and Drug Administration (FDA) for use as biotechnology drugs and vaccines, with another 370 in clinical trials. Proteins tested to date come from both prokaryotic and eukaryotic sources and are quite varied in both structure and function. Optimizing expression and/or activity for a wide variety of proteins involves the testing and usage of a multitude of factors which can affect transcription, translation, solubility and stability of the protein of interest. Factors which can affect protein expression are environmental (e.g., temperature or nutrients), host cell specific (e.g., protease deficiency or chaperone overexpression), plasmid specific (e.g., type of promoter, secretion signal), or sequence specific (e.g., altered codon usage for specific host).

One factor that can affect the expression and activity level of a recombinant protein is the genetic makeup of the plasmid or expression construct comprising the recombinant gene. This includes the regulatory sequences required to direct the expression and secretion of the protein. For example, a strong promoter that is functional within the host cell in which a protein is produced may be required.

Another factor that can affect the expression and activity level of a recombinant protein is the polynucleotide sequence encoding the protein. Alterations to the native sequence, such as modifying the sequence to reflect the codon usage of a particular host cell, can result in enhanced expression levels.

Provided herein are methods and compositions for the heterologous expression of a protein of interest.

### BRIEF SUMMARY OF THE INVENTION

Improved expression constructs and methods for identifying such constructs are provided. The expression constructs comprise a combination of regulatory elements and coding sequences that provide for optimal expression of a polypeptide of interest in an expression system.

The methods involve selecting an optimal expression construct from a population of expression constructs. The members of the population of expression constructs comprise identical type IIS restriction sites, and at least two members of the population comprise at least one distinct regulatory element or regulatory sequence. One or more members of the population may further comprise a distinct polynucleotide sequence encoding a polypeptide of interest compared to other members of the population. In this manner, expression constructs that provide for optimal expression of a particular polypeptide of interest can be identified. Further provided are methods for the use of the optimal expression construct for the production of the polypeptide of interest.

Methods to generate a population of expression vectors using a polynucleotide synthesis cyclic amplification reaction are also provided. Members of the population of expression vectors comprise identical type IIS restriction sites, and at least two members of the population comprise at least one distinct regulatory element or regulatory sequence. The vectors can be used to generate the population of expression constructs comprising a polynucleotide sequence encoding the polypeptide of interest.

### BRIEF DESCRIPTION OF THE FIGURES

FIG. 1 depicts an embodiment of the present invention, wherein an expression construct is produced. SapIa: SapI restriction enzyme recognition site with 3′ flanking sequence A; SapIb: SapI restriction enzyme recognition site with 3′ flanking sequence B; 5′UTR: 5′ untranslated region; RBS: ribosome binding site; sigseq: signal sequence; Hyb site: hybridization sequence (i.e., complementary sequence).

FIG. 2 depicts another embodiment of the present invention, wherein an expression construct is produced that is comprised of a polynucleotide sequence comprising two coding regions encoding two polypeptides of interest, with a bidirectional transcriptional termination sequence disposed between the two coding regions. SapIa: SapI restriction enzyme recognition site with 3′ flanking sequence A; SapIb: SapI restriction enzyme recognition site with 3′ flanking sequence B; 5′UTR: 5′ untranslated region; RBS: ribosome binding site; sigseq: signal sequence.

FIG. 3 shows the bidirectional terminators with restriction cohesive ends cloned into an expression vector between the promoter and ribosome biding site (RBS).

FIG. 4 shows the constructs that were tested for each bidirectional terminator.

FIG. 5 shows the expression results for each construct. Relative fluorescence (RF) was measured by spectrofluorimetry. COP-GFP expression from DC454 carrying either pDOW2942 (A), pDOW2943 (Ar), pDOW2950 (B), pDOW2951 (Br), pDOW2952 (C), pDOW2953 (Cr), pDOW2947 (BrA), or pDOW2954 (ArB) is shown. The letter P indicates the positive control (DC454/pDOW1344) and letter N=negative control (DC454/pDOW1169). I0, I24, and I48 represent 0 hour, 24 hours, and 48 hours post induction respectively. The letters A, Ar, B, Br, C, Cr, BrA, and ArB correlate with plasmid constructed as shown in FIG. 4.

FIG. 6 depicts the scheme for developing constructs having a bidirectional transcription terminator. The A and Ar represent plasmids pDOW2942 and pDOW2943; the B and Br refer to pDOW2950 and pDOW2951; the C and Cr indicate pDOW2952 and pDOW2953; and the BrA and ArB refer pDOW2947 and pDOW2954 respectively.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

It is to be noted that the term “a” or “an” entity refers to one or more of that entity; for example, “a regulatory element” is understood to represent one or more regulatory elements. As such, the terms “a” (or “an”), “one or more,” and “at least one” can be used interchangeably herein.

### I. General Overview

When expressing recombinant polypeptides, optimal expression and/or activity of the polypeptide may be influenced by the particular combination of regulatory elements that control the expression of the polypeptide. In addition, modifications to the polypeptide or polynucleotide sequence encoding the polypeptide can enhance the expression and/or activity of the polypeptide. Identification of the combination of regulatory elements necessary for optimized expression and function of each polypeptide of interest requires the laborious process of constructing and testing multiple expression constructs, each with a different set of regulatory elements and/or polypeptide coding sequences. Therefore, simplification of the procedures required to identify a set of regulatory elements that optimizes the expression and/or function of a polypeptide of interest or variant thereof would be advantageous. Thus, the present invention provides methods and compositions useful for the generation and screening of populations of expression constructs comprising a plurality of combinations of regulatory elements, as well as a plurality of polypeptide variants to aid in the development and identification of those expression constructs that are useful for optimal expression, secretion, and/or activity of polypeptide(s) of interest.

Methods of the present invention use type IIS restriction enzymes to generate expression constructs. Similar to other type II restriction enzymes commonly used in cloning techniques, type IIS restriction enzymes recognize and associate with a particular polynucleotide sequence, the recognition sequence. However, unlike other type II restriction enzymes, type IIS restriction enzymes do not cleave the polynucleotide chain within the recognition sequence. Rather, type IIS restriction enzymes cleave sequences outside of the recognition sequence. This unique characteristic of type IIS restriction enzymes allows one to clone or subclone polynucleotide sequences without the introduction of extraneous sequences, referred to as seamless cloning. This type of cloning is especially useful for those embodiments of the invention in which a signal sequence or peptide tag is fused in frame with the polypeptide of interest.

The present invention is directed to a method of generating a population of expression constructs that can be transformed into a host cell to express a polypeptide of interest. The methods of the invention allow for the seamless cloning of a coding region encoding a polypeptide of interest into an expression vector comprising a regulatory element. Populations of expression vectors and constructs can be generated with the methods of the present invention, and at least two members of the population of expression constructs can be comprised of a unique combination of regulatory elements and/or polypeptide coding sequences. Transformation of host cells with these expression constructs, followed by an assessment of the levels of expression and activity of the polypeptide of interest, can lead to the identification of those regulatory elements that optimize the expression, secretion and/or activity of a particular polypeptide of interest.

### II. Compositions

The present invention provides compositions comprising a population of expression vectors and expression constructs comprising a plurality of combinations of regulatory elements and/or regulatory sequences, as well as a plurality of different polynucleotide sequences encoding a polypeptide of interest. The population of expression constructs can be screened for the identification of those expression constructs that are useful for optimized expression, secretion, and/or activity of the polypeptide(s) of interest.

Specifically, the compositions of the present invention are comprised of a population of expression vectors, wherein the members of the population comprise at least one type IIS restriction enzyme recognition site adjacent to a regulatory element, wherein members of the population of expression vectors comprise identical type IIS restriction enzyme recognition sites, and wherein the regulatory element and/or regulatory sequence is distinct in at least two members of the population of expression vectors.

By “expression construct” or “expression vector” is intended a DNA molecule, particularly a plasmid nucleotide sequence, that has been generated through the arrangement of certain polynucleotide sequence elements, wherein the DNA molecule is operable in a host cell of interest (e.g., capable of expressing a polynucleotide encoding a polypeptide of interest, and/or capable of replicating in the host cell). The elements can include vector sequences, regulatory elements, and a polynucleotide sequence comprising at least one coding region encoding a polypeptide of interest. Although the terms “expression vector” and “expression construct” can be used interchangeably to describe a DNA molecule that comprises a polynucleotide sequence encoding a polypeptide of interest, as used herein, an “expression vector” may not comprise a coding sequence for a polypeptide of interest, whereas an “expression construct” will comprise a coding sequence for a polypeptide of interest.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, that has been removed from its native environment. Examples of an isolated polynucleotide include recombinant polynucleotides maintained in heterologous host cells or purified (partially or substantially) polynucleotides in solution. Isolated polynucleotides or nucleic acids according to the present invention further include such molecules produced synthetically. Isolated polynucleotides can also include isolated expression vectors, expression constructs, or populations thereof “Polynucleotide” can also refer to amplified products of itself, as in a polymerase chain reaction. The “polynucleotide” may contain modified nucleic acids, such as phosphorothioate, phosphate, ring atom modified derivatives, and the like. The “polynucleotide” of the invention may be a naturally occurring polynucleotide (i.e., one existing in nature without human intervention), or a recombinant polynucleotide (i.e., one existing only with human intervention).

For the purposes of the present invention, a “coding sequence for a polypeptide of interest” or “coding region for a polypeptide of interest” refers to the polynucleotide sequence that encodes that polypeptide. As used herein, the terms “encoding” or “encoded” when used in the context of a specified nucleic acid mean that the nucleic acid comprises the requisite information to direct translation of the nucleotide sequence into a specified polypeptide. The information by which a polypeptide is encoded is specified by the use of codons. The “coding region” or “coding sequence” is the portion of the nucleic acid that consists of codons that can be translated into amino acids. Although a “stop codon” or “translational termination codon” (TAG, TGA, or TAA) is not translated into an amino acid, it may be considered to be part of a coding region. Likewise, a transcription initiation codon (ATG) may or may not be considered to be part of a coding region. However, any sequences flanking the coding region, for example promoters, ribosome binding sites, transcriptional terminators, introns, and the like, are not considered to be part of the coding region. In some embodiments, however, while not considered part of the coding region per se, these regulatory sequences and any other regulatory sequence, particularly signal sequences or sequences encoding a peptide tag, may be part of the polynucleotide sequence encoding the polypeptide of interest. Thus, a polynucleotide sequence encoding a polypeptide of interest comprises the coding sequence and optionally any sequences flanking the coding region that contribute to expression, secretion, and/or isolation of the polypeptide of interest.

The term “population” is intended a group in which members of the group share one or more characteristics. The compositions of the invention comprise a population of expression vectors, wherein the members of the population comprise identical type IIS restriction enzyme recognition sites. However, at least two members have distinct regulatory elements or distinct regulatory sequence(s), or both, and the members of the population of expression vectors may comprise identical or non-identical vector sequences. In some embodiments, at least 3, at least 5, at least 8, at least 10, at least 15, at least 20, at least 30, or at least 50 members or more have distinct regulatory element(s). By “distinct” is intended non-identical when compared to other members of the population. In one embodiment, members of the population comprise distinct regulatory elements. For example, one member may comprise a secretion signal sequence whereas another member may comprise a tag sequence. In this and other embodiments, it is recognized that the absence of one or more regulatory elements from a member of the population makes that member distinct from any other member that comprises that regulatory element. In another embodiment, members of the population comprise the same regulatory elements, but the sequence of that element is different. For example, two members are considered distinct when each comprises the secretion signal, but one comprises secretion signal sequence “A” and the other comprises secretion signal sequence “B”. For the purposes of the present invention, the term “regulatory element” is used to describe the type of regulatory sequence (e.g., a ribosomal binding site element, a secretion signal element, a tag element, etc), and the term “regulatory sequence” refers to the actual nucleotide or amino acid sequence of the regulatory element (e.g., sequence “A” or sequence “B” exemplified above).

A. Vector Sequences

Expression vectors of the present invention comprise vector sequences. By “vector sequence” is intended a polynucleotide sequence that comprises an origin of replication to ensure maintenance of the vector and to, if desirable, provide amplification within the host, and one or more phenotypic selectable markers. Suitable hosts for transformation in accordance with the present disclosure include both eukaryotic and prokaryotic hosts. Prokaryotic hosts include all species within the genera Pseudomonas, particularly the host cell strain of P. fluorescens. In some embodiments, vector sequences of the expression vectors or expression constructs can be derived from any vector known in the art. While any vector or polynucleotide sequence comprising an origin of replication is useful in the present invention, in some embodiments, the vector sequences are derived from an expression plasmid, wherein the expression plasmid comprises regulatory sequences.

Vectors are known in the art for expressing recombinant proteins in host cells, and any of these may be used in the present invention. Such vectors include, e.g., plasmids, cosmids, and phage expression vectors. Examples of useful plasmid vectors include, but are not limited to, the expression plasmids pBBR1MCS, pDSK519, pKT240, pML122, pPS10, RK2, RK6, pRO1600, and RSF1010. Other examples of such useful vectors include those described by, e.g.: N. Hayase, in Appl. Envir. Microbiol. 60(9):3336-42 (September 1994); A. A. Lushnikov et al., in Basic Life Sci. 30:657-62 (1985); S. Graupner & W. Wackemagel, in Biomolec. Eng. 17(1):11-16. (October 2000); H. P. Schweizer, in Curr. Opin. Biotech. 12(5):439-45 (October 2001); M. Bagdasarian & K. N. Timmis, in Curr. Topics Microbiol. Immunol. 96:47-67 (1982); T. Ishii et al., in FEMS Microbiol. Lett. 116(3):307-13 (Mar. 1, 1994); I. N. Olekhnovich & Y. K. Fomichev, in Gene 140(1):63-65 (Mar. 11, 1994); M. Tsuda & T. Nakazawa, in Gene 136(1-2):257-62 (Dec. 22, 1993); C. Nieto et al., in Gene 87(1):145-49 (Mar. 1, 1990); J. D. Jones & N. Gutterson, in Gene 61(3):299-306 (1987); M. Bagdasarian et al., in Gene 16(1-3):237-47 (December 1981); H. P. Schweizer et al., in Genet. Eng. (NY) 23:69-81 (2001); P. Mukhopadhyay et al., in J. Bact. 172(1):477-80 (January 1990); D. O. Wood et al., in J. Bact. 145(3):1448-51 (March 1981); and R. Holtwick et al., in Microbiology 147(Pt 2):337-44 (February 2001).

Further examples of expression plasmids that can be useful in the present invention include those listed in Table 1 as derived from the indicated replicons.

TABLE 1
Examples of Useful Expression Vectors

| Replicon | Vector(s) |
|---|---|
| PPS10 | PCN39, PCN51 |
| RSF1010 | PKT261-3 |
| | PMMB66EH |
| | PEB8 |
| | PPLGN1 |
| | PMYC1050 |
| RK2/RP1 | PRK415 |
| | PJB653 |
| PRO1600 | PUCP |
| | PBSP |

The expression plasmid, RSF1010, is described, e.g., by F. Heffron et al., in Proc. Nat'l Acad. Sci. USA 72(9):3623-27 (September 1975), and by K. Nagahari & K. Sakaguchi, in J. Bact. 133(3):1527-29 (March 1978). Plasmid RSF1010 and derivatives thereof are particularly useful vectors in the present invention. Exemplary, useful derivatives of RSF1010, which are known in the art, include, e.g., pKT212, pKT214, pKT231 and related plasmids, and pMYC1050 and related plasmids (see, e.g., U.S. Pat. Nos. 5,527,883 and 5,840,554 to Thompson et al.), such as, e.g., pMYC1803. Plasmid pMYC1803 is derived from the RSF1010-based plasmid pTJS260 (see U.S. Pat. No. 5,169,760 to Wilcox), which carries a regulated tetracycline resistance marker and the replication and mobilization loci from the RSF1010 plasmid. Other exemplary useful vectors include those described in U.S. Pat. No. 4,680,264 to Puhler et al.

In one embodiment, vector sequences of the expression vectors of the present invention comprise sequences from RSF1010 or a derivative thereof. In still another embodiment, vector sequences from pMYC1050 or a derivative thereof, or pMYC4803 or a derivative thereof comprise the expression vectors of the present invention. In yet another embodiment, the population of expression vectors of the invention is comprised of sequences from the vector pDOW1169, or a derivative thereof.

Plasmid vectors can be maintained in the host cell by inclusion of a selection marker gene in the plasmid. This may be an antibiotic resistance gene(s), where the corresponding antibiotic(s) is added to the fermentation medium, or any other type of selection marker gene known in the art, e.g., a prototrophy-restoring gene where the plasmid is used in a host cell that is auxotrophic for the corresponding trait, e.g., a biocatalytic trait such as an amino acid biosynthesis or a nucleotide biosynthesis trait, or a carbon source utilization trait. In some embodiments, the polynucleotide encoding the polypeptide of interest serves as the selectable marker gene, where host cells are selected based on the expression of the polypeptide of interest.

B. Restriction Enzymes

Expression vectors and expression constructs of the invention comprise at least one type IIS restriction enzyme recognition site. Restriction enzymes or restriction endonucleases are proteins that are able to cleave or break double-stranded DNA sequences. These enzymes recognize and bind to or associate with a particular target polynucleotide sequence (i.e., restriction enzyme recognition site) and break or cleave the polynucleotide chains within or near to the recognition site. By “restriction enzyme recognition site” is intended the polynucleotide sequence that can be bound or “recognized” by a restriction enzyme. Restriction enzymes can be grouped based on similar characteristics. In general there are three major types or classes: I, II (including IIS) and III. Class I enzymes cut at a somewhat random site from the enzyme recognition sites (see Old and Primrose, Principles of Gene Manipulation, Blackwell Sciences, Inc., Cambridge, Mass., (1994)). Class III restriction enzymes are rare and are not commonly used in molecular biology.

Type II enzymes are the restriction enzymes most frequently used in molecular biology techniques. The type II recognition sequences can be continuous or interrupted. Type IIS restriction enzymes generally recognize non-palindromic sequences and cleave outside of their recognition site. (see, Szybalski et al. (1985) Gene 40: 169-173; Szybalski et al., Gene 100: 13-26 (1991); and Ausubel et al., eds. (1995) Current Protocols in Molecular Biology (Greene Publishing and Wiley-Interscience, New York)), herein incorporated by reference in its entirety. See Roberts et al. (2007) Nucleic Acids Research 35:D269-D270 and the REBASE website at rebase.nev.com/cgi-bin/asymmlist, herein incorporated by reference in their entireties, for a listing of type IIS restriction enzymes and the corresponding restriction enzyme recognition sites.

For the purposes of the present invention, a “type IIS restriction enzyme recognition site” or a “type IIS restriction site” or a “type IIS restriction enzyme recognition sequence” is a polynucleotide sequence that is recognized by a type IIS restriction enzyme. The recognition and subsequent association with the restriction enzyme recognition site by the type IIS enzyme results in cleavage of a polynucleotide sequence having the recognition site by the type IIS enzyme. The cleavage occurs outside of the recognition sequence. It is further noted that the term “type IIS restriction enzyme recognition site” can encompass a type IIS restriction enzyme site that is a complement or reverse complement of the described recognition site for that particular enzyme.

Expression vectors and expression constructs of the invention can comprise a type IIS restriction enzyme recognition site that is recognized by a type IIS restriction enzyme that cleaves DNA molecules, leaving overhanging ends or blunt ends. However, type IIS restriction enzymes that cleave outside of their recognition site, creating 5′ or 3′ overhanging sequence are especially useful in the present invention. By “overhanging end” or “overhanging sequences” is intended a terminus of a double-stranded DNA molecule which has one or more unpaired nucleotides in one of the two strands. The “overhanging end” can be either on the 5′ end or the 3′ end of a single strand of DNA. Conversely, “blunt end” is intended a terminus of a double-stranded DNA molecule with no unpaired nucleotides in either strand.

Examples of type IIS restriction enzymes that cleave DNA molecules, leaving overhanging ends (either 5′ or 3′; referred to herein as “overhanging-end type IIS restriction enzymes”), that are useful in the present invention include, but are not limited to, AarI, Acc36I, AceIII, AclWI, AcuI, AjuI, AloI, AlwI, Alw26I, AlwXI, AsuHPI, BaeI, Bbr7I, BbsI, BbvI, BbvII, Bbv161I, BccI, Bce83I, BceAI, Beef', BciVI, BcgI, Bco5I, Bco116I, BcoKI, BfiI, BfuAI, BfuI, BinI, Bli736I, Bme585I, BmrI, BmuI, BpiI, BpmI, BpuAI, BpuEI, BpuSI, BsaI, BsaXI, Bsc91I, BscAI, Bse3DI, BseGI, BseKI, BseMI, BseMII, BseRI, BseXI, BseZI, BsgI, BslFI, BsmAI, BsmBI, BsmFI, Bso31I, BsoMAI, Bsp24I, Bsp423I, BspBS31I, BspCNI, BspIS41, BspKT51, BspLU11III, BspMI, BspPI, BspQI, BspST5I, BspTNI, BspTS5141, BsrD1, Bst6I, Bst12I, Bst19I, Bst71I, BstBS32I, BstF5I, BstFZ438I, BstGZ53I, BstH9I, BstMAI, BstOZ616I, BstV1I, BstV2I, Bst31TI, BstT35I, Bsu6I, BtgZI, BtsCI, BtsI, BveI, CjeI, CjePI, CseI, CspCI, CstMI, EacI, Eam1104I, EarI, EciI, Eco31I, Eco57I, Eco57MI, EcoA4I, EcoO44I, Esp3I, FaqI, FauI, FokI, GsuI, HgaI, Hin4I, Hin4II, HphI, HpyAV, HpyC1I, Ksp632I, LguI, LweI, MboII, MmeI, MnlI, NcuI, NmeAIII, PciSI, PhaI, PleI, PpiI, PpsI, PsrI, RleAI, SapI, SfaNI, SmuI, Sth132I, StsI, TaqII, TsoI, TspDTI, TspGWI, TstI, Tth1111I, VpaK321, and the like. In particular, type IIS restriction enzymes that cleave a DNA sequence 3′ to the recognition site find use in the present invention. In some embodiments of the present invention, the type HS restriction enzyme recognition site present in the expression vectors or expression constructs is SapI. In another embodiment, the expression vector comprises at least two type IIS restriction enzyme recognition sites. The at least two recognition sites may be identical (e.g., recognized by the same type IIS enzyme) or non-identical (e.g., recognized by two different type HS enzymes).

In other embodiments, expression vectors and expression constructs of the present invention can be comprised of type IIS restriction enzyme recognition sites that are recognized by type IIS restriction enzymes that cleave DNA molecules, leaving blunt ends (referred to herein as “blunt-end type IIs restriction enzymes”). Such restriction enzymes include, but are not limited to, MlyI, SchI, and SspD5I. It will be further appreciated by a person of ordinary skill in the art that new type IIS restriction enzymes are continually being discovered and may be readily adapted for use in the subject invention.

C. Regulatory Elements

Expression vectors and expression constructs of the present invention comprise regulatory elements adjacent to the type IIS restriction enzyme recognition site. By “adjacent” or “adjacent to,” as used herein, is intended within less than about 250 nucleotides of the recognition site. In some embodiments, the regulatory element is less than about 200 nucleotides, less than about 150, less than about 100, less than about 75, less than about 50, less than about 40, less than about 30, less than about 20, less than about 10, less than about 5, 4, 3, 2, or 1 nucleotides from the type IIS restriction enzyme recognition site. In some of these embodiments, the regulatory element is immediately adjacent to the type IIS restriction enzyme recognition site, with no nucleotides disposed in between the two sequences.

By “regulatory elements” is intended elements (e.g., nucleotide and/or amino acid sequences) that control the expression, secretion, and/or activity of a polypeptide of interest. Regulatory elements can include transcription control elements, translation control elements, and polynucleotide sequences that encode peptide tags or signal peptides. Transcription control elements that are operably associated with one or more coding regions can regulate the transcription of a coding region that it is operably associated therewith. Examples of transcription control elements include promoters, enhancers, operators, repressors, and transcription termination signals. An “operable association” is when a coding region for a polypeptide of interest is associated with one or more regulatory elements in such a way as to place expression of the polypeptide of interest under the influence or control of the regulatory element(s). Two DNA fragments (such as a polypeptide coding region and a promoter associated therewith) are “operably associated” or “operably linked” if induction of promoter function results in the transcription of mRNA encoding the desired polypeptide of interest and if the nature of the linkage between the two DNA fragments does not interfere with the ability of the expression regulatory elements to direct the expression of the polypeptide of interest or interfere with the ability of the DNA template to be transcribed. Thus, a promoter region would be operably associated with a polynucleotide sequence encoding a polypeptide of interest if the promoter was capable of effecting transcription of that polynucleotide sequence.

The promoters used in accordance with the present invention may be constitutive promoters or regulated promoters. Examples of regulated promoters include those that are cell-specific and direct substantial transcription of the DNA only in predetermined cells, inducible promoters, wherein the activity is induced in the presence of a certain molecule, and those promoters that regulate the transcription of the gene product in a temporal manner. Common examples of useful regulated promoters include those of the family derived from the lac promoter (i.e. the lacZ promoter), especially the tac and trc promoters described in U.S. Pat. No. 4,551,433 to DeBoer, as well as Ptac16, Ptac17, PtacII, PlacUV5, and the T7lac promoter. In one embodiment, the promoter is not derived from the host cell organism. In certain embodiments, the promoter is derived from an E. coli organism.

Common examples of non-lac-type promoters useful in expression vectors and expression constructs according to the present invention include, e.g., those listed in Table 2.

TABLE 2
Examples of non-lac Promoters

| Promoter | Inducer |
|---|---|
| P_{R} | High temperature |
| P_{L} | High temperature |
| Pm | Alkyl- or halo-benzoates |
| Pu | Alkyl- or halo-toluenes |
| Psal | Salicylates |

See, e.g.: J. Sanchez-Romero & V. De Lorenzo (1999) Genetic Engineering of Nonpathogenic Pseudomonas strains as Biocatalysts for Industrial and Environmental Processes, in Manual of Industrial Microbiology and Biotechnology (A. Demain & J. Davies, eds.) pp. 460-74 (ASM Press, Washington, D.C.); H. Schweizer (2001) Vectors to express foreign genes and techniques to monitor gene expression for Pseudomonads, Current Opinion in Biotechnology, 12:439-445; and R. Slater & R. Williams (2000) The Expression of Foreign DNA in Bacteria, in Molecular Biology and Biotechnology (J. Walker & R. Rapley, eds.) pp. 125-54 (The Royal Society of Chemistry, Cambridge, UK)). A promoter having the nucleotide sequence of a promoter native to the selected bacterial host cell may also be used to control expression of the transgene encoding the target polypeptide, e.g, a Pseudomonas anthranilate or benzoate operon promoter (Pant, Pben). Tandem promoters may also be used in which more than one promoter is covalently attached to another, whether the same or different in sequence, e.g., a Pant-Pben tandem promoter (interpromoter hybrid) or a Plac-Plac tandem promoter, or whether derived from the same or different organisms.

Some regulated promoters utilize promoter regulatory proteins in order to control transcription of the gene of which the promoter is a part. Where such regulated promoters are used, a corresponding promoter regulatory protein will also be part of an expression construct according to the present invention. Examples of promoter regulatory proteins include: activator proteins, e.g., E. coli catabolite activator protein, MalT protein; AraC family transcriptional activators; repressor proteins, e.g., E. coli LacI proteins; and dual-function regulatory proteins, e.g., E. coli NagC protein. Many regulated-promoter/promoter-regulatory-protein pairs are known in the art.

Promoter regulatory proteins interact with an effector compound, i.e. a compound that reversibly or irreversibly associates with the regulatory protein so as to enable the protein to either release or bind to at least one DNA transcription regulatory region of the gene that is under the control of the promoter, thereby permitting or blocking the action of a transcriptase enzyme in initiating transcription of the gene. Effector compounds are classified as either inducers or co-repressors, and these compounds include native effector compounds and gratuitous inducer compounds. Many regulated-promoter/promoter-regulatory-protein/effector-compound trios are known in the art. Although an effector compound can be used throughout the cell culture or fermentation, in a preferred embodiment in which a regulated promoter is used, after growth of a desired quantity or density of host cell biomass, an appropriate effector compound is added to the culture to directly or indirectly result in expression of the desired gene(s) encoding the protein or polypeptide of interest.

By way of example, where a lac family promoter is utilized, a lad gene can also be present in the system. The lad gene, which is (normally) a constitutively expressed gene, encodes the Lac repressor protein (LacD protein) which binds to the lac operator of these promoters. Thus, where a lac family promoter is utilized, the lad gene can also be included and expressed in the expression system. In the case of the lac promoter family members, e.g., the tac promoter, the effector compound is an inducer, preferably a gratuitous inducer such as IPTG (isopropyl-D-1-thiogalactopyranoside, also called “isopropylthiogalactoside”).

Other transcription control elements that find utility in the present invention include, but are not limited to, those that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Transcription of the DNA encoding polypeptides of interest may be increased by inserting an enhancer sequence into the vector or plasmid. Typical enhancers are cis-acting elements of DNA, usually from about 10 to 300 by in size that act on the promoter to increase its transcription. Examples include various Pseudomonas enhancers.

Regulatory elements can also include translation control elements, which are known to those of ordinary skill in the art. These include, but are not limited to, ribosome binding sites, translation initiation codons (ATG) and termination codons (TAG, TGA, or TAA), and elements derived from picornaviruses (particularly an internal ribosome entry site, or IRES, also referred to as a CITE sequence). Useful RBSs can be obtained from any of the species useful as host cells in expression systems according to the present invention, preferably from the selected host cell. Many specific and a variety of consensus RBSs are known, e.g., those described in and referenced by D. Frishman et al., Starts of bacterial genes: estimating the reliability of computer predictions, Gene 234(2):257-65 (8 Jul. 1999); and B. E. Suzek et al., A probabilistic method for identifying start codons in bacterial genomes, Bioinformatics 17(12):1123-30 (December 2001). In addition, either native or synthetic RBSs may be used, e.g., those described in: EP 0207459 (synthetic RBSs); O. Ikehata et al., Primary structure of nitrile hydratase deduced from the nucleotide sequence of a Rhodococcus species and its expression in Escherichia coli, Eur. J. Biochem. 181(3):563-70 (1989) (native RBS sequence of AAGGAAG).

Further examples of methods, vectors, and transcription and translation control elements, and other elements useful in the present invention are described in, e.g.: U.S. Pat. No. 5,055,294 to Gilroy and U.S. Pat. No. 5,128,130 to Gilroy et al.; U.S. Pat. No. 5,281,532 to Rammler et al.; U.S. Pat. Nos. 4,695,455 and 4,861,595 to Barnes et al.; U.S. Pat. No. 4,755,465 to Gray et al.; and U.S. Pat. No. 5,169,760 to Wilcox.

The expression vectors or expression constructs of the invention may comprise regulatory elements, such as a polynucleotide sequence that encodes for a signal peptide. In some embodiments, the expression constructs of the present invention comprise a secretion signal sequence that, when expressed, functions in Gram negative bacteria to transport the polypeptide into the periplasmic space or the extracellular medium. Gram-negative bacteria have evolved numerous systems for the active export of proteins across their dual membranes. These routes of secretion include, e.g.: the ABC (Type I) pathway, the Path/Fla (Type III) pathway, and the Path/Vir (Type IV) pathway for one-step translocation across both the plasma and outer membrane; the Sec (Type II), Tat, MscL, and Holins pathways for translocation across the plasma membrane; and the Sec-plus-fimbrial usher porin (FUP), Sec-plus-autotransporter (AT), Sec-plus-two partner secretion (TPS), Sec-plus-main terminal branch (MTB), and Tat-plus-MTB pathways for two-step translocation across the plasma and outer membranes. In one such embodiment in which the polypeptide of interest is to be expressed by a Gram-negative bacterium, the secretion signal sequence allows for translocation of the polypeptide across the bacterial inner membrane into the perisplasmic space. Such signal sequences include a Sec, a Tat, a MscL, and a Holins signal sequence, or any other signal sequence known to one of ordinary skill in the art that when expressed, is able to direct the transport of a polypeptide into the periplasmic space of a Gram-negative bacterium.

In some of these embodiments, the expression construct of the invention further comprises a coding sequence for an autotransporter, a two partner secretion system, a main terminal branch system or a fimbrial usher porin that when expressed, directs the polypeptide to be translocated across the outer membrane into the extracellular medium. Examples of signal sequences useful in the present invention include, but are not limited to, the sequences disclosed in U.S. Pat. No. 5,348,867; U.S. Pat. No. 6,329,172; PCT Publication No. WO 96/17943; PCT Publication No. WO 02/40696; U.S. Application Publication 2003/0013150; PCT Publication No. WO 03/079007; U.S. Publication No. 2003/0180937; U.S. Publication No. 2003/0064435; and, PCT Publication No. WO 00/59537; U.S. Pat. No. 5,914,254; U.S. Pat. No. 4,963,495; European Patent No. 0 177 343; U.S. Pat. No. 5,082,783; PCT Publication No. WO 89/10971; U.S. Pat. No. 6,156,552; U.S. Pat. Nos. 6,495,357; 6,509,181; 6,524,827; 6,528,298; 6,558,939; 6,608,018; 6,617,143; U.S. Pat. Nos. 5,595,898; 5,698,435; and 6,204,023; U.S. Pat. No. 6,258,560; PCT Publication Nos. WO 01/21662, WO 02/068660 and U.S. Application Publication 2003/0044906; U.S. Pat. No. 5,641,671; European Patent No. EP 0 121 352; and the signal sequences disclosed in U.S. App. No. 60/887,476, Attorney Docket No. 043292/319802, filed on Jan. 31, 2007, entitled “A phosphate binding protein leader sequence for increased expression.” In one embodiment, the signal sequences useful in the methods of the invention comprise the Sec secretion system signal sequences (see, Agarraberes and Dice (2001) Biochim Biophys Acta. 1513:1-24; Muller et al. (2001) Prog Nucleic Acid Res Mol. Biol. 66:107-157; and U.S. Patent Application Nos. 60/887,476 and 60/887,486, filed Jan. 31, 2007, each of which is herein incorporated by reference in its entirety). In one such embodiment, the signal sequence is the phosphate binding protein (pbp) leader sequence (or derivatives thereof) described in U.S. Patent Application No. 60/887,476, Attorney Docket No. 043292/319802, filed Jan. 31, 2007, entitled “A phosphate binding leader sequence for increased expression.”

In other embodiments, the expression vectors or expression constructs of the invention comprise a polynucleotide sequence that encodes a secretory or signal peptide, which directs the secretion of the polypeptide of interest in a eukaryotic cell, or any other polynucleotide sequence encoding a protease cleavage site. For example, proteins secreted by mammalian cells have a signal peptide or secretory leader sequence that is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. Such sequences are useful in the present invention. In certain embodiments, the native signal peptide is used, or a functional derivative of that sequence that retains the ability to direct the secretion of the polypeptide with which it is operably associated. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

In some embodiments, the expression construct comprises a polynucleotide coding sequence that encodes a polypeptide of interest as well as a polynucleotide sequence that encodes a peptide tag that is useful in the identification, separation, purification, and/or isolation of the polypeptide of interest. The polynucleotide sequence encoding such a peptide tag can be adjacent to the coding region for the polypeptide of interest or adjacent to the leader or signal sequence, if applicable. Thus, in some embodiments, the expression construct can comprise both a polynucleotide sequence that encodes a peptide tag useful in the identification, separation, purification, and/or isolation of the polypeptide of interest and a polynucleotide sequence that encodes a signal sequence or leader that targets the polypeptide of interest to the periplasmic space or the extracellular medium. In one embodiment, this peptide tag sequence allows for purification of the protein. The tag sequence can be an affinity tag, such as a hexa-histidine affinity tag. In another embodiment, the affinity tag can be a glutathione-S-transferase molecule. The tag can also be a fluorescent molecule, such as yellow-fluorescent protein (YFP) or green-fluorescent protein (GFP), or analogs of such fluorescent proteins. The tag can also be a portion of an antibody molecule, or a known antigen or ligand for a known binding partner useful for purification.

D. Polypeptides of Interest

The methods and compositions of the present invention are useful for producing properly processed polypeptides of interest in a cell expression system. In some embodiments, the compositions comprise expression constructs comprising polynucleotide sequences encoding a polypeptide of interest. The polynucleotide sequence encoding the polypeptide of interest may comprise a naturally occurring coding sequence (i.e., one existing in nature without human intervention). Alternatively, the polynucleotide sequence may be a synthetic or recombinant coding sequence (i.e., one existing only with human intervention).

As discussed supra, the polynucleotide sequence encoding the polypeptide of interest can further comprise regulatory elements, including a signal sequence or a coding sequence for a peptide tag. In such embodiments, the polypeptide, when produced, also includes a signal peptide that targets the protein to the periplasmic space. In some of these embodiments, the polypeptide comprises a signal peptide that directs the transport of the protein into the extracellular medium. In other embodiments, the signal sequence or peptide tag sequence are present within the expression vector of the invention, leading to the expression of a polypeptide including a peptide tag. Other suitable regulatory elements are discussed elsewhere herein.

As used herein, the term “polypeptide of interest” or “protein of interest” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” or “polypeptide of interest” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids.

The polypeptide of interest can be of any species and of any size. However, in certain embodiments, the protein or polypeptide of interest is a therapeutically useful protein or polypeptide. In some embodiments, the protein can be a mammalian protein, for example a human protein, and can be, for example, a growth factor, a cytokine, a chemokine or a blood protein. The protein or polypeptide of interest can be processed in a similar manner to the native protein or polypeptide. In certain embodiments, the protein or polypeptide of interest is less than 100 kD, less than 50 kD, or less than 30 kD in size. In certain embodiments, the protein or polypeptide of interest is a polypeptide of at least about 5, 10, 15, 20, 30, 40, 50, 100, 200, 500, 1000, or 2000 amino acids.

Extensive sequence information required for molecular genetics and genetic engineering techniques is widely publicly available. Access to complete nucleotide sequences of mammalian, as well as human, genes, cDNA sequences, amino acid sequences and genomes can be obtained from GenBank at the website www.ncbi.nlm.nih.gov/Entrez. Additional information can also be obtained from GeneCards, an electronic encyclopedia integrating information about genes and their products and biomedical applications from the Weizmann Institute of Science Genome and Bioinformatics (bioinformatics.weizmann.ac.il/cards), nucleotide sequence information can be also obtained from the EMBL Nucleotide Sequence Database (www.ebi.ac.uk/embl) or the DNA Databank or Japan (DDBJ, www.ddbi.nig.ac.jp). Additional sites for information on amino acid sequences include Georgetown's protein information resource website (www.pir.georgetown.edu) and Swiss-Prot (au.expasy.org/sprot/sprot-top.html).

Examples of polypeptides that can be expressed in this invention include molecules such as renin, a growth hormone, including human growth hormone; bovine growth hormone; growth hormone releasing factor; parathyroid hormone; thyroid stimulating hormone; lipoproteins; α-1-antitrypsin; insulin A-chain; insulin B-chain; proinsulin; thrombopoietin; follicle stimulating hormone; calcitonin; luteinizing hormone; glucagon; clotting factors such as factor VIIIC, factor IX, tissue factor, and von Willebrands factor; anti-clotting factors such as Protein C; atrial naturietic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; a serum albumin such as human serum albumin; mullerian-inhibiting substance; relaxin A-chain; relaxin B-chain; prorelaxin; mouse gonadotropin-associated polypeptide; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as brain-derived neurotrophic factor (BDNF), neurotrophin-3, -4, -5, or -6 (NT-3, NT-4, NT-5, or NT-6), or a nerve growth factor such as NGF-13; cardiotrophins (cardiac hypertrophy factor) such as cardiotrophin-1 (CT-1); platelet-derived growth factor (PDGF); fibroblast growth factor such as aFGF and bFGF; epidermal growth factor (EGF); transforming growth factor (TGF) such as TGF-alpha and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; anti-HER-2 antibody; superoxide dismutase; T-cell receptors; surface membrane proteins; decay accelerating factor; viral antigen such as, for example, a portion of the AIDS envelope; transport proteins; homing receptors; addressins; regulatory proteins; antibodies; and fragments of any of the above-listed polypeptides.

In certain embodiments, the polypeptide can be selected from the group consisting of IL-1, IL-1a, IL-1b, IL-2, IL-3, IL-4, IL-5, IL-6, IL-7, IL-8, IL-9, IL-10, IL-11, IL-12, IL-12elasti, IL-13, IL-15, IL-16, IL-18, IL-18BPa, IL-23, IL-24, VIP, erythropoietin, GM-CSF, G-CSF, M-CSF, platelet derived growth factor (PDGF), MSF, FLT-3 ligand, EGF, fibroblast growth factor (FGF; e.g., α-FGF (FGF-1), β-FGF (FGF-2), FGF-3, FGF-4, FGF-5, FGF-6, or FGF-7), insulin-like growth factors (e.g., IGF-1, IGF-2); tumor necrosis factors (e.g., TNF, Lymphotoxin), nerve growth factors (e.g., NGF), vascular endothelial growth factor (VEGF); interferons (e.g., IFN-α, IFN-β, IFN-γ); leukemia inhibitory factor (LIF); ciliary neurotrophic factor (CNTF); oncostatin M; stem cell factor (SCF); transforming growth factors (e.g., TGF-α, TGF-β1, TGF-β2, TGF-β3); TNF superfamily (e.g., LIGHT/TNFSF14, STALL-1/TNFSF13B (BLy5, BAFF, THANK), TNFalpha/TNFSF2 and TWEAK/TNFSF12); or chemokines (BCA-1/BLC-1, BRAK/Kec, CXCL16, CXCR3, ENA-78/LIX, Eotaxin-1, Eotaxin-2/MPIF-2, Exodus-2/SLC, Fractalkine/Neurotactin, GROalpha/MGSA, HCC-1, I-TAC, Lymphotactin/ATAC/SCM, MCP-1/MCAF, MCP-3, MCP-4, MDC/STCP-1/ABCD-1, MIP-1.quadrature., MIP-1.quadrature., MIP-2.quadrature./GRO.quadrature., MIP-3.quadrature./Exodus/LARC, MIP-3/Exodus-3/ELC, MIP-4/PARC/DC-CK1, PF-4, RANTES, SDF1, TARC, or TECK).

In one embodiment of the present invention, the polypeptide of interest can be a multi-subunit protein or polypeptide. Multisubunit proteins that can be expressed include homomeric and heteromeric proteins. The multisubunit proteins may include two or more subunits, that may be the same or different. For example, the protein may be a homomeric protein comprising 2, 3, 4, 5, 6, 7, 8, 9, 10, 11, 12 or more subunits. The protein also may be a heteromeric protein including 2, 3, 4, 5, 6, 7, 8, 9, 10, 11, 12, or more subunits. Exemplary multisubunit proteins include: receptors including ion channel receptors; extracellular matrix proteins including chondroitin; collagen; immunomodulators including MHC proteins, full chain antibodies, and antibody fragments; enzymes including RNA polymerases, and DNA polymerases; and membrane proteins.

In another embodiment, the polypeptide of interest can be a blood protein. The blood proteins expressed in this embodiment include but are not limited to carrier proteins, such as albumin, including human and bovine albumin, transferrin, recombinant transferrin half-molecules, haptoglobin, fibrinogen and other coagulation factors, complement components, immunoglobulins, enzyme inhibitors, precursors of substances such as angiotensin and bradykinin, insulin, endothelin, and globulin, including alpha, beta, and gamma-globulin, and other types of proteins, polypeptides, and fragments thereof found primarily in the blood of mammals. The amino acid sequences for numerous blood proteins have been reported (see, S. S. Baldwin (1993) Comp. Biochem Physiol. 106b:203-218), including the amino acid sequence for human serum albumin (Lawn, L. M., et al. (1981) Nucleic Acids Research, 9:6103-6114.) and human serum transferrin (Yang, F. et al. (1984) Proc. Natl. Acad. Sci. USA 81:2752-2756).

In another embodiment, the polypeptide of interest can be a recombinant enzyme or co-factor. The enzymes and co-factors expressed in this embodiment include, but are not limited to, aldolases, amine oxidases, amino acid oxidases, aspartases, B12 dependent enzymes, carboxypeptidases, carboxyesterases, carboxylyases, chemotrypsin, CoA requiring enzymes, cyanohydrin synthetases, cystathione synthases, decarboxylases, dehydrogenases, alcohol dehydrogenases, dehydratases, diaphorases, dioxygenases, enoate reductases, epoxide hydrases, fumerases, galactose oxidases, glucose isomerases, glucose oxidases, glycosyltrasferases, methyltransferases, nitrile hydrases, nucleoside phosphorylases, oxidoreductases, oxynitrilases, peptidases, glycosyltransferases, peroxidases, enzymes fused to a therapeutically active polypeptide, tissue plasminogen activator; urokinase, reptilase, streptokinase; catalase, superoxide dismutase; DNase, amino acid hydrolases (e.g., asparaginase, amidohydrolases); carboxypeptidases; proteases, trypsin, pepsin, chymotrypsin, papain, bromelain, collagenase; neuramimidase; lactase, maltase, sucrase, and arabinofuranosidases.

In another embodiment, the polypeptide of interest can be a single chain, Fab fragment and/or full chain antibody or fragments or portions thereof. A single-chain antibody can include the antigen-binding regions of antibodies on a single stably-folded polypeptide chain. Fab fragments can be a piece of a particular antibody. The Fab fragment can contain the antigen binding site. The Fab fragment can contain 2 chains: a light chain and a heavy chain fragment. These fragments can be linked via a linker or a disulfide bond.

In other embodiments, the polypeptide of interest is a protein that is active at a temperature from about 20 to about 42° C. In one embodiment, the protein is active at physiological temperatures and is inactivated when heated to high or extreme temperatures, such as temperatures over 65° C.

In one embodiment, the polypeptide of interest is a protein that is active at a temperature from about 20 to about 42° C. and/or is inactivated when heated to high or extreme temperatures, such as temperatures over 65° C.

1. Polynucleotide and Polypeptide Variants

The coding sequence for the protein or polypeptide of interest can be a native coding sequence for the polypeptide of interest. Naturally occurring allelic variants can be identified with the use of well-known molecular biology techniques, such as polymerase chain reaction (PCR) and hybridization techniques known in the art. Variant polynucleotides also include synthetically derived polynucleotides that have been generated, for example, by using site-directed or other mutagenesis strategies but which still encode the polypeptide having the desired biological activity.

For example, the polynucleotide coding regions encoding the polypeptide of interest may be adjusted based on the codon usage of a host organism. Codon usage or codon preference is well known in the art. The selected coding sequence may be modified by altering the genetic code thereof to match that employed by the host cell, and the codon sequence thereof may be enhanced to better approximate that employed by the host. Genetic code selection and codon frequency enhancement may be performed according to any of the various methods known to one of ordinary skill in the art, e.g., oligonucleotide-directed mutagenesis. Useful internet resources to assist in this process include, e.g.: (1) the Codon Usage Database of the Kazusa DNA Research Institute (2-6-7 Kazusa-kamatari, Kisarazu, Chiba 292-0818 Japan) and available at www.kazusa.or.jp/codon; and (2) the Genetic Codes tables available from the NCBI Taxonomy database at www.ncbi.nlm.nih.gov/Taxonomy/Utils/wprintgc.cgi?mode=c. For example, Pseudomonas species are reported as utilizing Genetic Code Translation Table 11 of the NCBI Taxonomy site, and at the Kazusa site as exhibiting the codon usage frequency of the table shown at www.kazusa.orip/codon/cgibin. It is recognized that the coding sequence for either the regulatory element, the polypeptide of interest described elsewhere herein, or both, can be adjusted for codon usage.

In those embodiments in which the polynucleotide sequence encoding the polypeptide of interest is introduced into the expression vector through the use of restriction enzymes, such as a type IIS restriction enzyme, the coding sequence of this polynucleotide sequence and/or the expression vector polynucleotide sequence may be modified to protect the sequences from unwanted digestion at restriction sites. Such modifications include changing the polypeptide coding sequence, the vector sequence, or both, by any mutagenesis or gene shuffling strategies known to one of ordinary skill in the art to remove or mutate restriction enzyme recognition sites, as well as the introduction of methylated nucleotides, such as 5-methyl-dCTP, within the sequence to protect the sequence from cleavage (Short, J. M. 1988, Nuc Acids Res 16:7583-7600; G. L. Costa, 1994, Strategies 7:8). Removal of the restriction sites from the population of expression vectors of the invention or the polynucleotide sequence encoding the polypeptide of interest, or both, will obviate the necessity of performing partial digestion reactions in order to avoid digesting either sequence at unwanted restriction sites. In some embodiments, the restriction sites within the polynucleotides are modified in such a way as to conserve the amino acid sequence of the polypeptide of interest, and/or any regulatory element of the construct, where applicable.

The skilled artisan will appreciate that changes can be introduced by further mutation of the polynucleotides of the invention, thereby leading to changes in the amino acid sequence of the encoded polypeptide(s) of interest. In such embodiments, the population of variant polypeptides can be screened, for example, for improved activity, secretion, and/or expression of the polypeptide, as described elsewhere herein. Thus, variant polypeptides can be created by introducing one or more substitutions, additions, or deletions into the corresponding polynucleotide coding region encoding the polypeptide of interest, such that one or more amino acid substitutions, additions or deletions are introduced into the encoded polypeptide of interest. Further mutations can be introduced by standard techniques, such as, for example, by: 1.) error-prone PCR (Leung et al., Techniques, 1:11-15 (1989); Zhou et al., Nucleic Acids Res., 19:6052-6052 (1991); Spee et al., Nucleic Acids Res., 21:777-778 (1993); Melnikov et al., Nucleic Acids Research, 27(4):1056-1062 (Feb. 15, 1999)); 2.) site directed mutagenesis (Coombs et al., Proteins, 259-311, 1 plate. Ed.: Angeletti, Ruth Hogue. Academic: San Diego, Calif. (1998)); 3.) in vivo mutagenesis; and 4.) “gene shuffling” (U.S. Pat. No. 5,605,793; U.S. Pat. No. 5,811,238; U.S. Pat. No. 5,830,721; and U.S. Pat. No. 5,837,458, hereby incorporated by reference). Additional methods for introducing nucleotides and/or amino acid substitutions are known in the art and encompassed herein.

The use of such variant polynucleotides encoding variant polypeptides of interest is also encompassed by the present invention. In some embodiments, the members of the population of expression vectors or expression constructs comprise identical polynucleotide sequences encoding a polypeptide of interest. In other embodiments, at least two members of the expression vector or expression construct population comprise distinct polynucleotide sequences encoding the same polypeptide of interest. In yet another embodiment, at least two members comprise distinct polynucleotide sequences encoding at least two variant polypeptides. For the purposes of the present invention, a “variant” polypeptide refers to a polypeptide that is at least about 50% identical, at least about 55% identical, at least about 60%, at least about 65%, at least about 70%, at least about 75%, at least about 80%, at least about 85%, at least about 90%, at least about 95%, at least about 96%, at least about 97%, at least about 98%, or at least about 99% identical to a reference polypeptide. A “reference polypeptide” refers to the parent sequence into which amino acid additions, substitutions, or deletions were introduced to create the variant polypeptide. This “variant polypeptide” designation will distinguish individual expression constructs encoding distinct polypeptides, as described above, from expression constructs that encode more than one polypeptide from the same construct, as discussed below. Generally, when the members of a population of expression vectors or expression constructs are non-identical, the members will comprise more than one variant polynucleotide sequence coding region that may or may not encode polypeptide variants.

2. Expression Constructs Comprising More than One Coding Region

In some embodiments, expression constructs of the present invention comprise a polynucleotide sequence comprising at least one coding region encoding a polypeptide of interest. In some of these embodiments, the polynucleotide sequence comprises a first coding region encoding a first polypeptide of interest and a second coding region encoding a second polypeptide of interest. In these embodiments, the first and second coding regions can be operably linked to a single promoter and are, therefore, co-transcribed, producing a dicistronic transcript representing coding information for both polypeptides. Where the constructs are designed for expression in a eukaryotic cell, the first and the second coding region may have an internal ribosome sequence (IRES) disposed between the two coding regions to allow for the separate translation of each of the coding regions within the single transcript. The presence of an IRES site between these coding regions permits the production of the second polypeptide of interest encoded by the second coding region by internal initiation of the translation of the dicistronic transcript. Any IRES sequences known in the art can be used in the present invention, particularly those of the picornaviruses.

In other embodiments wherein two polypeptides of interest are expressed by a single expression construct, the first and the second coding regions can each be operably linked to a set of separate regulatory elements, including a promoter and transcription termination sequence. In these embodiments, the two coding regions are transcribed and translated separately. Where the two coding regions are separately transcribed from different promoters, the promoter and coding region for each polypeptide may be present in the construct in the same orientation, and a transcription termination sequence is present between the two coding regions. In this manner, each coding region is transcribed from separate promoters, and each coding region will have its own transcription termination sequence (e.g., promoter 1-coding region 1-terminator-promoter 2-coding region 2).

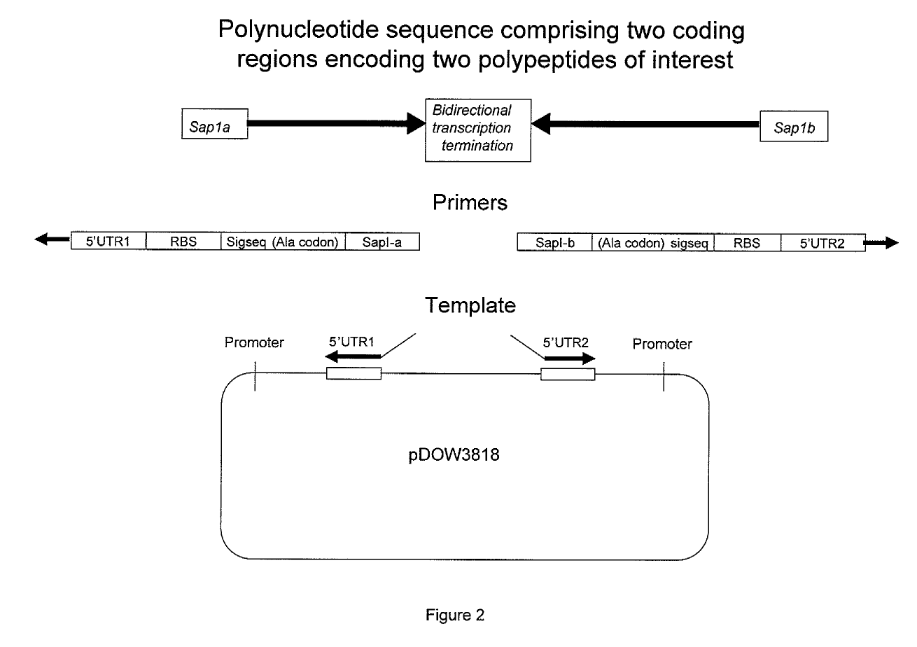

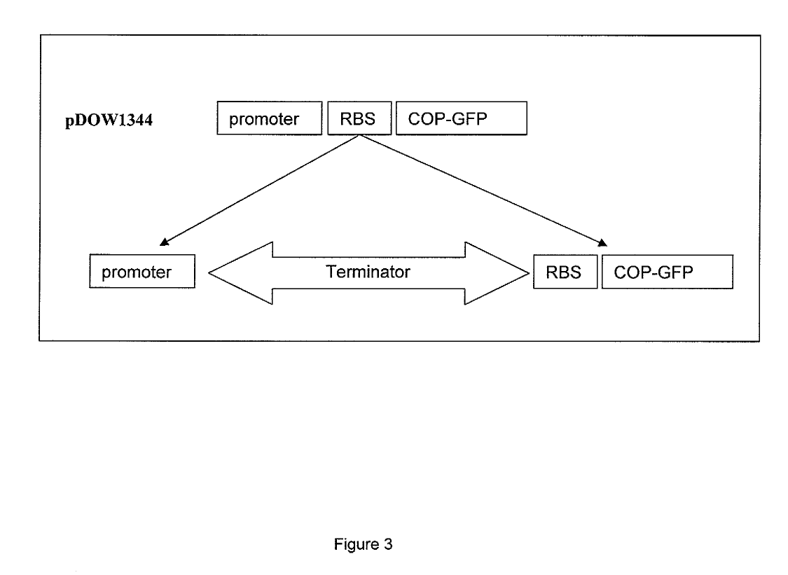

In other embodiments, the first and the second coding regions are separated by a bidirectional transcription termination sequence, each coding region is operably associated with a separate set of regulatory elements (e.g., promoters), and the coding regions are separately transcribed and translated. In these embodiments, the coding regions and operably associated regulatory elements are oriented in such a manner as to allow the transcription of the two coding regions to proceed towards the bidirectional transcription termination sequence. See, for example, FIG. 2.

In various embodiments, the bidirectional transcription termination sequence comprises the nucleotide sequence set forth in SEQ ID NOs: 7, 8 or 9. Additional bidirectional termination sequences are known in the art. See, for example, Schollmeier and Gaertner (1985) Nucleic Acids Research 13(12):4227-4237, which is herein incorporated by reference in its entirety. Other bidirectional terminators can be identified using methods known in the art. See, for example, Kingsford et al. (2007) Genome Biology 8:R22 and Ermolaeva et al. (2000) J Mol Biol 301(1):27-33, each of which is herein incorporated by reference in its entirety.

### II. Methods

A. Methods for Generating a Population of Expression Vectors

The present invention discloses methods for the generation of a population of expression vectors. The method comprises performing a polynucleotide synthesis cyclic amplification reaction comprising (a) a polynucleotide template comprising a target polynucleotide; (b) a population of first oligonucleotide primers comprising a first complementary sequence that is complementary to a first region of the target polynucleotide; and (c) a population of second oligonucleotide primers comprising a second complementary sequence that is complementary to a second region of the target polynucleotide, such that the population of first and the population of second oligonucleotide primers allow for the amplification of the target polynucleotide. According to the methods of the invention, at least one member of the population of the first oligonucleotide primers, at least one member of the population of second oligonucleotide primers, or at least one member of the population of both the first and second oligonucleotide primers comprise (in the 5′ to 3′ direction) a type IIS restriction enzyme recognition site, a regulatory element adjacent to the recognition site, and the sequence complementary to the target polynucleotide. Amplification of the target polynucleotide using the primers disclosed herein result in the incorporation of one or more type IIS restriction site(s) and one or more regulatory elements into the polynucleotide template to generate an expression vector.

In some embodiments, the resulting population of expression vectors comprises linear vectors, and it may be desirable to circularize the vectors to facilitate introduction and maintenance of the population of expression vectors in a suitable host cell. For example, it may be desirable to produce large quantities of the population in a host cell capable of replicating the vector. Depending on the design of the primers used to generate the expression vectors, the vectors may be circularized by ligating the blunt-ended polynucleotide synthesis product of the amplification reaction. One of skill in the art will recognize that additional steps may be necessary to facilitate the ligation and circularization of the population of expression vectors.

1. Polynucleotide Synthesis Cyclic Amplification Reaction

Methods for the generation of a population of expression vectors include performing a polynucleotide synthesis cyclic amplification reaction. By “polynucleotide synthesis cyclic amplification reaction” is intended any reaction whereby a polynucleotide sequence is amplified. While, the person skilled in the art of nucleic acid amplification knows the existence of rapid amplification procedures such as ligase chain reaction (LCR), transcription-based amplification systems (TAS), self-sustained sequence replication (3SR), nucleic acid sequence-based amplification (NASBA), strand displacement amplification (SDA) and branched DNA (bDNA) (Persing et al, 1993. Diagnostic Molecular Microbiology: Principles and Applications, American Society for Microbiology, Washington, D.C.), polymerase chain reaction (PCR) is the most widely used method of nucleic acid amplification and will be further described herein. However, the scope of this invention is not limited to the use of amplification by PCR, but rather includes the use of any rapid nucleic acid amplification methods or any other procedures which may be useful for the amplification of the polynucleotide template.

Numerous different PCR protocols known in the art and exemplified herein can be directly applied or adapted for the method of the present invention. Generally, in PCR, a target polynucleotide sequence is amplified by reaction with at least one oligonucleotide primer or pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (e.g., the target polynucleotide sequence which is herein referred to as the amplification product or the “polynucleotide synthesis product”). The amplification cycle is repeated one or more times to increase the concentration of the polynucleotide synthesis product.

### a. Oligonucleotide Primers

In the present invention, oligonucleotide primers are used in the cyclic amplification of the target polynucleotide. As used herein, a “primer” refers to a type of oligonucleotide having or containing a sequence complementary to a region of a polynucleotide template, which hybridizes to the polynucleotide template through base pairing. The term “oligonucleotide” refers to a short polynucleotide, typically less than or equal to 250 nucleotides long (e.g., between 5 and 250, between 10 to 100, or between 15 to 50 nucleotides in length). However, as used herein, the term is also intended to encompass longer or shorter polynucleotide chains.

As used herein, the term “polynucleotide template” refers to a polynucleotide sequence that serves as a pattern for the synthesis of a DNA molecule in a polynucleotide synthesis cyclic amplification reaction, such as PCR. A polynucleotide template of the invention may be comprised of a naturally occurring polynucleotide (i.e., one existing in nature without human intervention), or a recombinant polynucleotide (i.e., one existing only with human intervention), including but not limited to genomic DNA, cDNA, plasmid DNA, total RNA, mRNA, tRNA, rRNA. In some embodiments, the polynucleotide template is a vector. In further embodiments, the polynucleotide template is an expression vector. Examples of vectors that can serve as polynucleotide templates in the cyclic amplification reactions of the invention include, but are not limited to, those vectors that are disclosed elsewhere herein. As used herein, the term “target polynucleotide” refers to the portion of a polynucleotide template that is to be amplified in a polynucleotide synthesis cyclic amplification reaction. A “target polynucleotide” of the present invention contains a known sequence of at least 20 nucleotides, at least 50 nucleotides, at least 100 nucleotides, at least 500 nucleotides, at least 1000 nucleotides, at least 2000 nucleotides, at least 3000 nucleotides, at least 5000, at least 8000 or more nucleotides.

The primers disclosed herein comprise a sequence complementary to a region of the target polynucleotide. As used herein, the term “complementary” refers to the concept of sequence complementarity between regions of two polynucleotide strands or between two regions of the same polynucleotide strand. A first region of a polynucleotide is complementary to a second region of the same or a different polynucleotide if, when the two regions are arranged in an antiparallel fashion, at least one nucleotide of the first region is capable of base pairing with a base of the second region. Therefore, it is not required for two complementary polynucleotides to base pair at every nucleotide position. For the purposes of the present invention, “complementary” may refer to a first polynucleotide that is 100% or “fully” complementary to a second polynucleotide and thus forms a base pair at every nucleotide position. “Complementary” may also refer to a first polynucleotide that is not 100% complementary (e.g., 90%, or 80% or 70% complementary) and contains mismatched nucleotides at one or more nucleotide positions. Therefore, a “complementary sequence” is a polynucleotide sequence that is complementary to another polynucleotide sequence. In the present invention, the population of first oligonucleotide primers and the population of second oligonucleotide primers comprise complementary sequences that are complementary to a first and a second region, respectively, of the target polynucleotide that is to be amplified in the cyclic amplification reaction.

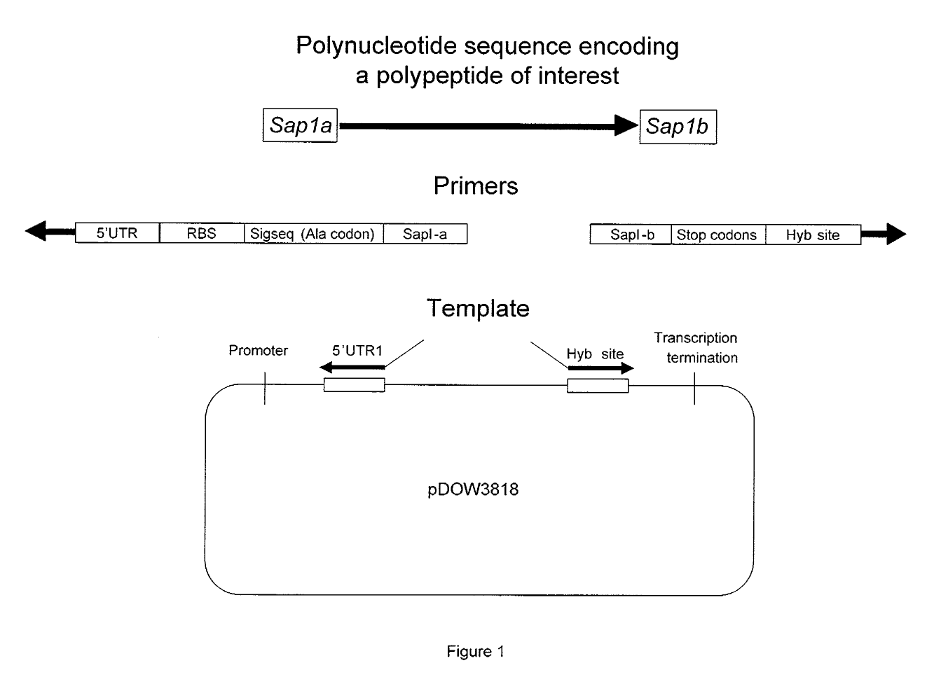

In some embodiments, complementarity between the primer and the target polynucleotide occurs across only a portion of the primer such that amplification of the template in the presence of the primer results in the incorporation of the region of the primer that is not complementary to the template into the expression vector. See, for example, the primer design in FIGS. 1 and 2. For hybridization to occur between the primer and the template polynucleotide, it will generally be necessary for the primer to comprise complementary sequence that is complementary to the template at least about 5, at least about 10, at least about 11, about 12, about 13, about 14, about 15, about 16, about 17, about 18, about 19, at least about 20 or more nucleotides.

As used herein, the term “hybridization” is used in reference to the pairing of complementary (including partially complementary) polynucleotide strands. Hybridization and the strength of hybridization (i.e., the strength of the association between polynucleotide strands) is impacted by many factors well known in the art including the degree of complementarity between the polynucleotides and the stringency of the hybridization conditions, such as the concentration of salts, the melting temperature (Tm) of the formed hybrid, the presence of other components (e.g., the presence or absence of polyethylene glycol), the molarity of the hybridizing strands and the G:C content of the polynucleotide strands. Unless otherwise specified, the strength of hybridization, or degree of complementarity, refers to hybridization under stringent conditions (i.e., hybridization in 50% formamide, 1 M NaCl, 1% SDS at 37° C., and a wash in 0.1×SSC at 60 to 65° C.).

As used herein, “Tm” and “melting temperature” are interchangeable terms which are the temperature at which 50% of a population of double-stranded polynucleotide molecules becomes dissociated into single strands. The equation for calculating the Tm of polynucleotides is well known in the art. For example, the Tm may be calculated by the following equation: Tₘ=69.3+0.41.times.(G+C) %−650/L, wherein L is the length of the probe in nucleotides. The Tm of a hybrid polynucleotide may also be estimated using a formula adopted from hybridization assays in 1 M salt, and commonly used for calculating Tm for PCR primers: [(number of A+T)×2° C.+(number of G+C)×4° C.], see, for example, Newton et al. (1997) PCR 2nd Ed. (Springer-Verlag, New York). Other more sophisticated computations exist in the art, which take structural as well as sequence characteristics into account for the calculation of Tm. A calculated Tm is merely an estimate; the optimum temperature is commonly determined empirically.

The population of first oligonucleotide primers comprises a first complementary sequence that is complementary to a first region of the target polynucleotide and the population of second oligonucleotide primers comprises a second complementary sequence that is complementary to a second region of the target polynucleotide. The orientation of the two populations of primers when hybridized or bound to the target polynucleotide is such to allow for the amplification of the target polynucleotide. For example, the population of first primers is able to hybridize with one strand of the target polynucleotide, whereas the population of second primers is able to hybridize with the opposite strand of the target polynucleotide, allowing (under conditions for a cyclic amplification reaction) for the amplification of the target polynucleotide. See, for example, FIG. 1.

At least one member of the first or the second population of oligonucleotide primers, or at least one member of each of the first and second populations of oligonucleotide primers, comprise (in the 5′ to 3′ direction) a type IIS restriction enzyme recognition site, a regulatory element adjacent to the type IIS restriction enzyme recognition sequence, and the complementary sequence that is complementary to a first region, second region, or both regions of the target polynucleotide.

The type IIS restriction enzyme recognition site can comprise a recognition site that is recognized by a type IIS restriction enzyme that cleaves DNA in a manner which leaves overhanging ends, or a recognition site that is recognized by a type IIS restriction enzyme that cleaves DNA in a manner which leaves blunt ends. Examples of type IIS restriction enzyme recognition sites include those recognized by the type IIS restriction enzymes that are disclosed elsewhere herein.

In various embodiments, at least one member of the population of first, second, or both primers comprises a regulatory element. Examples of regulatory elements include those that are disclosed elsewhere herein. In some embodiments, the term “regulatory element” also refers to a sequence that is in the reverse complement orientation. For example, where a primer sequence is the reverse complement of the “coding” or the “sense” strand of the target polynucleotide, it will be understood that the sequence of the regulatory element will also be the reverse complement of any regulatory element otherwise defined herein.

The primers of the present invention can be prepared using techniques known in the art, including, but not limited to, cloning and digestion of the appropriate sequences and direct chemical synthesis.

Chemical synthesis methods that can be used to make the primers of the present invention, include, but are not limited to, the phosphotriester method described by Narang et al., Methods in Enzymology, 68:90 (1979), the phosphodiester method disclosed by Brown et al., Methods in Enzymology, 68:109 (1979), the diethylphosphoramidate method disclosed by Beaucage et al., Tetrahedron Letters, 22:1859 (1981) and the solid support method described in U.S. Pat. No. 4,458,066. The use of an automated oligonucleotide synthesizer to prepare synthetic oligonucleotide primers of the present invention is also contemplated herein. Additionally, if desired, the primers can be labeled using techniques known in the art and described below.

### b. Cyclic Amplification Reaction Conditions

Methods for setting up a cyclic amplification reaction are well known to those skilled in the art. While the present invention is not limited to the use of the polymerase chain reaction (PCR) method of cyclic amplification, exemplary conditions for PCR are described herein for the purposes of describing a suitable means for performing the steps of the invention. The PCR reaction mixture minimally comprises the polynucleotide template and oligonucleotide primers in combination with suitable buffers, salts, and the like, and an appropriate concentration of a nucleic acid polymerase. As used herein, “nucleic acid polymerase” refers to an enzyme that catalyzes the polymerization of nucleoside triphosphates. Generally, the enzyme will initiate synthesis at the 3′-end of the primer annealed to the target sequence, and will proceed in the 5′-direction along the template until synthesis terminates. An appropriate concentration includes one which catalyzes this reaction in the presently described methods. Known DNA polymerases include, for example, E. coli DNA polymerase I, T7 DNA polymerase, Thermus thermophilus (Tth) DNA polymerase, Bacillus stearothermophilus DNA polymerase, Thermococcus litoralis DNA polymerase, Thermus aquaticus (Taq) DNA polymerase and Pyrococcus furiosus (Pfu) DNA polymerase.

In addition to the above components, the reaction mixture produced in the subject methods includes primers and deoxyribonucleoside triphosphates (dNTPs). Each primer (first and second) is present at about 10 to about 500 nM, or about 25 to about 400 nM, or about 50 to about 300 nM, or about 250 nM.

Usually the reaction mixture will further comprise four different types of dNTPs corresponding to the four-naturally occurring nucleoside bases, i.e. dATP, dTTP, dCTP and dGTP. In the subject methods, each dNTP will typically be present in an amount ranging from about 10 to 5000 μM, usually from about 20 to 1000 μM, about 100 to 800 μM, or about 300 to 600 μM.

The PCR reaction mixture further includes an aqueous buffer medium that includes a source of monovalent ions, a source of divalent cations and a buffering agent. Any convenient source of monovalent ions, such as potassium chloride, potassium acetate, ammonium acetate, potassium glutamate, ammonium chloride, ammonium sulfate, and the like may be employed. The divalent cation may be magnesium, manganese, zinc and the like, where the cation will typically be magnesium. Any convenient source of magnesium cation may be employed, including magnesium chloride, magnesium acetate, and the like. The amount of magnesium present in the buffer may range from 0.5 to 10 mM, but will preferably range from about 1 to about 6 mM, or about 3 to about 5 mM. Representative buffering agents or salts that may be present in the buffer include Tris, Tricine, HEPES, MOPS and the like, where the amount of buffering agent will typically range from about 5 to 150 mM, usually from about 10 to 100 mM, and more usually from about 20 to 50 mM, where in certain preferred embodiments the buffering agent will be present in an amount sufficient to provide a pH ranging from about 6.0 to 9.5, or about pH 8.0. Other agents which may be present in the buffer medium include chelating agents, such as EDTA, EGTA and the like.

In preparing the reaction mixture, the various constituent components may be combined in any convenient order. For example, the buffer may be combined with primer, polymerase and then the template polynucleotide, or all of the various constituent components may be combined at the same time to produce the reaction mixture.

Alternatively, commercially available premixed reagents can be utilized in the methods of the invention according to the manufacturer's instructions, or modified to improve reaction conditions (e.g., modification of buffer concentration, cation concentration, or dNTP concentration, as necessary).

Following preparation of the reaction mixture, the reaction mixture is subjected to primer extension reaction conditions, i.e., conditions that permit for polymerase mediated primer extension by addition of nucleotides to the end of the primer molecule using the template strand as a template. In many embodiments, the primer extension reaction conditions are amplification conditions, which conditions include a plurality of reaction cycles, where each reaction cycle comprises: (1) a denaturation step, (2) an annealing step, and (3) a polymerization step. The number of reaction cycles will vary depending on the application, but will usually be at least 15, more usually at least 20 and may be as high as 60 or higher, where the number of different cycles will typically range from about 20 to 40. For methods where more than about 25, usually more than about 30 cycles are performed, it may be convenient or desirable to introduce additional polymerase into the reaction mixture such that conditions suitable for enzymatic primer extension are maintained.

The denaturation step comprises heating the reaction mixture to an elevated temperature and maintaining the mixture at the elevated temperature for a period of time sufficient for any double stranded or hybridized nucleic acid present in the reaction mixture to dissociate. For denaturation, the temperature of the reaction mixture will usually be raised to, and maintained at, a temperature ranging from about 85° C. to 100° C., usually from about 90° C. to 98° C. and more usually from about 93° C. to 96° C., for a period of time ranging from about 3 to 120 sec, usually from about 5 to 30 sec.

Following denaturation, the reaction mixture will be subjected to conditions sufficient for primer annealing to the polynucleotide template present in the mixture, and for polymerization of nucleotides to the primer ends in a manner such that the primer is extended in a 5′ to 3′ direction using the nucleic acid to which it is hybridized as a template. The temperature to which the reaction mixture is lowered to achieve these conditions will usually be chosen to provide optimal efficiency and specificity, and will generally range from about 50° C. to 75° C., usually from about 55° C. to 70° C. and more usually from about 60° C. to 68° C., more particularly around 62° C. Annealing conditions will be maintained for a period of time ranging from about 15 sec to 30 min, usually from about 20 sec to 5 min, or about 30 sec to 1 minute, or about 43 seconds.

This step can optionally comprise one of each of an annealing step and an extension step with variation and optimization of the temperature and length of time for each step. In a 2-step annealing and extension, the annealing step is allowed to proceed as above. Following annealing of primer to the polynucleotide template, the reaction mixture will be further subjected to conditions sufficient to provide for polymerization of nucleotides to the primer ends as above. To achieve polymerization conditions, the temperature of the reaction mixture will typically be raised to or maintained at a temperature ranging from about 65° C. to 75° C., usually from about 67° C. to 73° C., and maintained for a period of time ranging from about 15 sec to 20 min, usually from about 30 sec to 5 min.

The above cycles of denaturation, annealing and polymerization may be performed using an automated device, typically known as a thermal cycler. Thermal cyclers that may be employed are described elsewhere herein as well as in U.S. Pat. Nos. 5,612,473; 5,602,756; 5,538,871; and 5,475,610, the disclosures of which are herein incorporated by reference.

Variations on the exact amounts of the various reagents and on the conditions for the PCR or other suitable amplification procedure (e.g., buffer conditions, cycling times, etc.) that lead to similar amplification results are known to those of skill in the art and are considered to be equivalents.

B. Methods for Generating a Population of Expression Constructs

The methods of the invention for the generation of a population of expression vectors can further comprise additional steps for the production of expression constructs comprising a polynucleotide sequence comprising at least one coding region encoding a polypeptide of interest (also referred to herein as a “polynucleotide sequence encoding a polypeptide of interest”).

In these embodiments, the method further comprises cleaving the population of expression vectors with a type IIS restriction enzyme that recognizes the type IIS restriction enzyme recognition site, thereby producing a population of cleaved expression vectors. The population of cleaved expression vectors is then ligated to a population of polynucleotide sequences encoding a polypeptide of interest, wherein the population of polynucleotide sequences is ligation-compatible with the population of cleaved expression vectors. Ligation of the cleaved expression vectors with the population of polynucleotide sequences encoding the polypeptide of interest produces a population of expression constructs.

By “ligation-compatible” is intended the termini of the polynucleotide sequence are capable of hybridizing with or being ligated to the termini of the cleaved expression vectors. In some embodiments, the population of cleaved expression vectors and the population of polynucleotide sequences encoding a polypeptide of interest each comprise at least one blunt end. In some of these embodiments, the blunt end of the cleaved expression vector is the result of cleavage with a blunt-end type IIS restriction enzyme. In these embodiments, the type IIS restriction enzyme can be selected from the group consisting of MlyI, SchI, and SspD5I. The blunt end of the polynucleotide sequence comprising a coding region can be generated through amplification reactions, such as PCR, or through the hybridization of two chemically synthesized oligonucleotides. Alternatively, the blunt end can be generated from the cleavage of the polynucleotide sequence with a restriction enzyme that leaves blunt ends, such as a type IIS restriction enzyme. In this embodiment, the polynucleotide sequence encoding the polypeptide of interest further comprises a blunt-end type IIS restriction enzyme recognition site situated such that cleavage of the polynucleotide with the type IIs restriction enzyme removes the restriction enzyme recognition site.

Another way to generate blunt ends is through the modification of overhanging ends that have been produced through restriction digestion (i.e., “cleavage”), amplification reactions or hybridization of chemically synthesized oligonucleotides. Such modifications are known in the art and include enzymatic removal of the overhanging ends or “filling in” the overhanging ends. Enzymes with 3′ to 5′ exonuclease activity, such as T4 DNA polymerase, can remove 3′ overhanging ends. In addition, single-strand nucleases, such as mung bean nuclease and S1 nuclease, can remove both 5′ and 3′ overhanging sequences, creating blunt ends. 5′ overhanging ends on a polynucleotide sequence can be “filled in” with DNA polymerases, such as the Klenow fragment of DNA polymerase I or T4 DNA polymerase.

In other embodiments, the population of expression vectors and the population of polynucleotide sequences encoding a polypeptide of interest each comprise an overhanging end. In some of these embodiments, the overhanging end is generated through the cleavage of the polynucleotide sequence with an overhanging-end type IIS restriction enzyme. In these embodiments, the overhanging-end type IIS restriction enzyme can be selected from the group consisting of AarI, Acc36I, AceIII, AclWI, AcuI, AjuI, AloI, AlwI, Alw26I, AlwXI, AsuHPI, BaeI, Bbr7I, BbsI, BbvI, BbvII, Bbv16II, BccI, Bce83I, BceAI, BcefI, BciVI, BcgI, Bco5I, Bco116I, BcoKI, BfiI, BfuAI, BfuI, BinI, Bli736I, Bme585I, BmrI, BmuI, BpiI, BpmI, BpuAI, BpuEI, BpuSI, BsaI, BsaXI, Bsc91I, BscAI, Bse3DI, BseGI, BseKI, BseMI, BseMII, BseRI, BseXI, BseZI, BsgI, BslFI, BsmAI, BsmBI, BsmFI, Bso31I, BsoMAI, Bsp24I, Bsp423I, BspBS31I, BspCNI, BspIS41, BspKT51, BspLU11III, BspMI, BspPI, BspQI, BspST5I, BspTNI, BspTS5141, BsrD1, Bst6I, Bst12I, Bst19I, Bst71I, BstBS32I, BstF5I, BstFZ438I, BstGZ53I, BstH9I, BstMAI, BstOZ616I, BstV1I, BstV2I, Bst31TI, BstT35I, Bsu6I, BtgZI, BtsCI, BtsI, BveI, CjeI, CjePI, CseI, CspCI, CstMI, EacI, Eam1104I, EarI, EciI, Eco31I, Eco57I, Eco57MI, EcoA4I, EcoO44I, Esp3I, FaqI, FauI, FokI, GsuI, HgaI, Hin4I, Hin4II, HphI, HpyAV, HpyC1I, Ksp632I, LguI, LweI, MboII, MmeI, MnlI, NcuI, NmeAIII, PciSI, PhaI, PleI, PpiI, PpsI, PsrI, RleAI, SapI, SfaNI, SmuI, Sth132I, StsI, TaqII, TsoI, TspDTI, TspGWI, TstI, Tth111II, and VpaK321.

In the embodiments of the invention wherein the cleaved expression vector and the polynucleotide sequence encoding the polypeptide of interest comprise an overhanging end, the sequences of the overhanging ends that are to be ligated together must be such that the sequence of the overhanging end on the cleaved expression vector is complementary to the sequence of the overhanging end on the polynucleotide sequence encoding the polypeptide of interest (thus creating “ligation-compatible” ends). In some of these embodiments, both termini of the cleaved expression vector and both termini of the polynucleotide sequence encoding the polypeptide of interest comprise ligation-compatible overhanging ends. In one embodiment, the overhanging ends on both termini of both molecules are identical. Therefore, ligation of the polynucleotide sequence into the cleaved expression vector can occur in a non-directional manner. In these embodiments, the population of cleaved expression vectors can be treated with a phosphatase enzyme, such as shrimp alkaline phosphatase or calf intestinal phosphatase, prior to ligation with the polynucleotide sequence encoding the polypeptide of interest to remove a phosphate group from the 5′ terminus of the cleaved expression vector sequence and prevent the two termini of the vector from ligating together (i.e., self-ligation).

In other embodiments in which both termini of the sequences to be ligated comprise overhanging ends, the overhanging ends within each polynucleotide sequence are non-identical, resulting from cleavage of non-identical cleavage sites. In this case, one overhanging end of the polynucleotide sequence encoding the polypeptide of interest is complementary to one overhanging end of the cleaved expression vector, and the other overhanging end of the polynucleotide sequence is complementary to the other overhanging end of the expression vector to allow the two sequences to hybridize to one another. This facilitates directional ligation of the polynucleotide sequence encoding the polypeptide of interest into the expression vector. One of skill in the art will recognize proper strategies for designing ligation-compatible polynucleotide sequences encoding the polypeptide of interest such that insertion of the polynucleotide sequence into the expression vector occurs in the proper orientation. Where a sequence is designed to insert in either direction (e.g., in blunt or overhanging ligations), it is also within the skill of the relevant artisan to screen or select for sequences inserted in the proper orientation.

In some embodiments, the polynucleotide sequence of the expression vectors of the invention may be modified using procedures described herein and well known to those of ordinary skill in the art to mutate or remove one or more type IIS restriction enzyme recognition sites from regions of the expression vector other than the restriction enzyme recognition sites introduced through the primers disclosed herein. This obviates the need to perform a partial digest reaction to avoid cleaving unwanted restriction sites within, rather than at the end(s) of, the expression vector for those embodiments of the invention. In addition, in those embodiments wherein the polynucleotide sequence encoding the polypeptide of interest is made ligation-compatible with the expression vectors of the invention through a restriction digestion, particularly with a type IIS restriction enzyme, the polynucleotide sequence can also be modified to mutate or remove unwanted recognition sites from the polypeptide coding sequence prior to restriction digestion of the DNA molecule.

As discussed elsewhere herein, the coding sequence for the polypeptide of interest may be a naturally occurring polynucleotide sequence (i.e., one existing in nature without human intervention), or may be a synthetic or recombinant polynucleotide sequence (i.e., one existing only with human intervention). The polynucleotide sequence comprising the coding sequence may further comprise type IIS restriction enzyme recognition sites (outside of the polypeptide coding region) that may be cleaved along with the expression vector to be made ligation-compatible with the vector, or the polynucleotide sequence may be synthesized to be ligation-compatible with the cleaved expression vector. For example, individual strands of the polynucleotide sequence can be chemically synthesized through any method known to one of ordinary skill in the art, followed by hybridization of the two complementary strands. The design of each strand is such that the hybridized double-stranded sequence is ligation-compatible with the cleaved expression vector (e.g., contains sequences on the 5′ and 3′ termini that are complementary to the corresponding ends of the cleaved vector). Methods for chemical synthesis of a polynucleotide sequence include the phosphotriester method described by Narang et al., Methods in Enzymology, 68:90 (1979), the phosphodiester method disclosed by Brown et al., Methods in Enzymology, 68:109 (1979), the diethylphosphoramidate method disclosed by Beaucage et al., Tetrahedron Letters, 22:1859 (1981) and the solid support method described in U.S. Pat. No. 4,458,066. The use of an automated synthesizer to prepare synthetic single-stranded polynucleotide sequences, which can then be hybridized to one another to form a double-stranded DNA is also contemplated herein. Alternatively, the polynucleotide sequence comprising a coding region can be derived through PCR amplification of a naturally occurring, synthetic, or recombinant polynucleotide. A naturally occurring or recombinant polynucleotide sequence can also be obtained through restriction digestion of the desired sequence.

C. Methods for Expressing a Polypeptide of Interest and Identifying an Expression Construct

Compositions of the invention comprising a population of expression vectors can further comprise a polynucleotide sequence encoding a polypeptide of interest. As discussed supra, the population of expression constructs comprises a plurality of different constructs that vary in the number or type of regulatory elements, or vary in the coding sequences for the regulatory elements (i.e., the “regulatory sequence”) or the polypeptide of interest, or any combination thereof. Such compositions are useful for the expression of a polypeptide of interest and for the selection of expression constructs that allow for sufficient levels of expression of the polypeptide of interest. Thus, in various embodiments, the present invention further comprises methods for the expression of polypeptides and selection of an expression construct that is optimized for the heterologous expression of the polypeptide of interest By “optimized” is intended that the expression construct contains a combination of regulatory elements and/or coding sequences sufficient for the expression and/or secretion of the polypeptide of interest in (or from) a particular host cell. Such constructs are considered to express/secrete the polypeptide at a “sufficient level” in the host cell. A sufficient level refers to the quantity and/or quality (e.g., activity) of the polypeptide of interest.

While exemplary values for each of these criteria are provided elsewhere herein, it is understood that a sufficient level of a polypeptide of interest can vary depending on the nature of the polypeptide of interest, as well as the intended use of the polypeptide. For example, it may be desirable under certain conditions to minimize the level of protein expression within a particular host cell, e.g., when high levels of expression are toxic to the cell. Under other conditions, however, it may be desirable to maximize protein expression in a cell, e.g., when large quantities of protein are needed. It is also understood that multiple different expression constructs sufficient for the expression of a polypeptide of interest may be identified using the methods described herein, and selection of a single optimal construct (if necessary) depends on the stringency of selection (such as the absolute quantity and/or quality of desired protein).

Specifically, the present invention is directed to methods of identifying an expression construct containing the combination of regulatory elements and/or coding sequences sufficient for the expression of a polypeptide of interest in a host cell. The methods comprise obtaining a population of expression constructs, wherein the members of the population comprise at least one type IIS restriction enzyme recognition site adjacent to a regulatory element and a polynucleotide sequence encoding a polypeptide of interest. The members of the population of expression constructs comprise identical type IIS restriction enzyme recognition sites, and at least two members of the population comprise distinct regulatory elements and/or regulatory sequences. In some embodiments, at least 3, at least 5, at least 8, at least 10, at least 15, at least 20, at least 30, or at least 50 or more members have distinct regulatory elements and/or regulatory sequences. According to the methods of the invention, the members of the population of expression constructs are introduced into a population of host cells, and the host cells are independently cultured under conditions that allow for the expression of a polypeptide of interest in at least one host cell. The host cells expressing sufficient levels of the polypeptide of interest are selected and the expression construct is isolated from the selected host cells to determine the combination of regulatory elements and coding sequences that lead to the sufficient expression of the polypeptide (e.g., by sequencing at least a portion of the construct).

1. Host Cells

According to the methods of the invention, the members of the population of expression constructs are introduced into a population of host cells. The host cells comprising at least one member of the population of expression constructs are then independently cultured under conditions that allow for the expression of a polypeptide of interest in at least one host cell. The term “introducing” or “transforming” in the context of a polynucleotide, for example, an expression construct, is intended to mean presenting to the host cell the polynucleotide in such a manner that the polynucleotide gains access to the interior of at least one host cell. Transformation of the host cells with the expression vectors or expression constructs disclosed herein may be performed using any transformation methodology known in the art, and the bacterial host cells may be transformed as intact cells or as protoplasts (i.e. including cytoplasts). Exemplary transformation methodologies include poration methodologies, e.g., electroporation, protoplast fusion, bacterial conjugation, and divalent cation treatment, e.g., calcium chloride treatment or CaCl/Mg2+ treatment, or other well known methods in the art. See, e.g., Morrison, J. Bact., 132:349-351 (1977); Clark-Curtiss & Curtiss, Methods in Enzymology, 101:347-362 (Wu et al., eds, 1983), Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

In one embodiment, the host cell can be any cell capable of producing a protein or polypeptide of interest. The most commonly used systems to produce proteins or polypeptides of interest include certain bacterial cells, particularly E. coli, because of their relatively inexpensive growth requirements and potential capacity to produce protein in large batch cultures. Yeasts are also used to express biologically relevant proteins and polypeptides, particularly for research purposes. Systems include Saccharomyces cerevisiae or Pichia pastoris. These systems are well characterized, provide generally acceptable levels of total protein expression and are comparatively fast and inexpensive. Insect cell expression systems have also emerged as an alternative for expressing recombinant proteins in biologically active form. In some cases, correctly folded proteins that are post-translationally modified can be produced. Mammalian cell expression systems, such as Chinese hamster ovary cells, have also been used for the expression of proteins or polypeptides of interest.

In another embodiment, the host cell is a plant cell, including, but not limited to, a tobacco cell, corn, a cell from an Arabidopsis species, potato or rice cell. In another embodiment, a multicellular organism is analyzed or is modified in the process, including but not limited to a transgenic organism. Techniques for analyzing and/or modifying a multicellular organism are generally based on techniques described for modifying cells described below.

In another embodiment, the host cell can be a prokaryote such as a bacterial cell including, but not limited to an Escherichia or a Pseudomonas species. Typical bacterial cells are described, for example, in “Biological Diversity: Bacteria and Archaeans”, a chapter of the On-Line Biology Book, provided by Dr M J Farabee of the Estrella Mountain Community College, Arizona, USA at the website www.emc.maricotpa.edu/faculty/farabee/BIOBK/BioBookDiversity. In certain embodiments, the host cell can be a Pseudomonad cell, and can typically be a P. fluorescens cell. In other embodiments, the host cell can also be an E. coli cell. In another embodiment, the host cell can be a eukaryotic cell, for example an insect cell, including but not limited to a cell from a Spodoptera, Trichoplusia, Drosophila or an Estigmene species, or a mammalian cell, including but not limited to a murine cell, a hamster cell, a monkey, a primate or a human cell.

In one embodiment, the host cell can be a member of any of the bacterial taxa. The cell can, for example, be a member of any species of eubacteria. The host can be a member of any one of the taxa: Acidobacteria, Actinobacteira, Aquificae, Bacteroidetes, Chlorobi, Chlamydiae, Choroflexi, Chrysiogenetes, Cyanobacteria, Deferribacteres, Deinococcus, Dictyoglomi, Fibrobacteres, Firmicutes, Fusobacteria, Gemmatimonadetes, Lentisphaerae, Nitrospirae, Planctomycetes, Proteobacteria, Spirochaetes, Thermodesulfobacteria, Thermomicrobia, Thermotogae, Thermus (Thermales), or Verrucomicrobia. In a embodiment of a eubacterial host cell, the cell can be a member of any species of eubacteria, excluding Cyanobacteria.

The bacterial host can also be a member of any species of Proteobacteria. A proteobacterial host cell can be a member of any one of the taxa Alphaproteobacteria, Betaproteobacteria, Gammaproteobacteria, Deltaproteobacteria, or Epsilonproteobacteria. In addition, the host can be a member of any one of the taxa Alphaproteobacteria, Betaproteobacteria, or Gammaproteobacteria, and a member of any species of Gammaproteobacteria.

In one embodiment of a Gamma Proteobacterial host, the host will be member of any one of the taxa Aeromonadales, Alteromonadales, Enterobacteriales, Pseudomonadales, or Xanthomonadales; or a member of any species of the Enterobacteriales or Pseudomonadales. In one embodiment, the host cell can be of the order Enterobacteriales, the host cell will be a member of the family Enterobacteriaceae, or may be a member of any one of the genera Erwinia, Escherichia, or Serratia; or a member of the genus Escherichia. Where the host cell is of the order Pseudomonadales, the host cell may be a member of the family Pseudomonadaceae, including the genus Pseudomonas. Gamma Proteobacterial hosts include members of the species Escherichia coli and members of the species Pseudomonas fluorescens.

Other Pseudomonas organisms may also be useful. Pseudomonads and closely related species include Gram-negative Proteobacteria Subgroup 1, which include the group of Proteobacteria belonging to the families and/or genera described as “Gram-Negative Aerobic Rods and Cocci” by R. E. Buchanan and N.E. Gibbons (eds.), Bergey's Manual of Determinative Bacteriology, pp. 217-289 (8th ed., 1974) (The Williams & Wilkins Co., Baltimore, Md., USA) (hereinafter “Bergey (1974)”). Table 3 presents these families and genera of organisms.

TABLE 3
Families and Genera Listed in the Part, “Gram-Negative
Aerobic Rods and Cocci” (in Bergey (1974))

| | |
|---|---|
| Family I. Pseudomomonaceae | Gluconobacter |
| | Pseudomonas |
| | Xanthomonas |
| | Zoogloea |
| Family II. Azotobacteraceae | Azomonas |
| | Azotobacter |
| | Beijerinckia |
| | Derxia |
| Family III. Rhizobiaceae | Agrobacterium |
| | Rhizobium |
| Family IV. Methylomonadaceae | Methylococcus |
| | Methylomonas |
| Family V. Halobacteriaceae | Halobacterium |
| | Halococcus |
| Other Genera | Acetobacter |
| | Alcaligenes |
| | Bordetella |
| | Brucella |
| | Francisella |
| | Thermus |

“Gram-negative Proteobacteria Subgroup 1” also includes Proteobacteria that would be classified in this heading according to the criteria used in the classification. The heading also includes groups that were previously classified in this section but are no longer, such as the genera Acidovorax, Brevundimonas, Burkholderia, Hydrogenophaga, Oceanimonas, Ralstonia, and Stenotrophomonas, the genus Sphingomonas (and the genus Blastomonas, derived therefrom), which was created by regrouping organisms belonging to (and previously called species of) the genus Xanthomonas, the genus Acidomonas, which was created by regrouping organisms belonging to the genus Acetobacter as defined in Bergey (1974). In addition hosts can include cells from the genus Pseudomonas, Pseudomonas enalia (ATCC 14393), Pseudomonas nigrifaciensi (ATCC 19375), and Pseudomonas putrefaciens (ATCC 8071), which have been reclassified respectively as Alteromonas haloplanktis, Alteromonas nigrifaciens, and Alteromonas putrefaciens. Similarly, e.g., Pseudomonas acidovorans (ATCC 15668) and Pseudomonas testosteroni (ATCC 11996) have since been reclassified as Comamonas acidovorans and Comamonas testosteroni, respectively; and Pseudomonas nigrifaciens (ATCC 19375) and Pseudomonas piscicida (ATCC 15057) have been reclassified respectively as Pseudoalteromonas nigrifaciens and Pseudoalteromonas piscicida. “Gram-negative Proteobacteria Subgroup 1” also includes Proteobacteria classified as belonging to any of the families: Pseudomonadaceae, Azotobacteraceae (now often called by the synonym, the “Azotobacter group” of Pseudomonadaceae), Rhizobiaceae, and Methylomonadaceae (now often called by the synonym, “Methylococcaceae”). Consequently, in addition to those genera otherwise described herein, further Proteobacterial genera falling within “Gram-negative Proteobacteria Subgroup 1” include: 1) Azotobacter group bacteria of the genus Azorhizophilus; 2) Pseudomonadaceae family bacteria of the genera Cellvibrio, Oligella, and Teredinibacter; 3) Rhizobiaceae family bacteria of the genera Chelatobacter, Ensifer, Liberibacter (also called “Candidatus Liberibacter”), and Sinorhizobium; and 4) Methylococcaceae family bacteria of the genera Methylobacter, Methylocaldum, Methylomicrobium, Methylosarcina, and Methylosphaera.

In another embodiment, the host cell is selected from “Gram-negative Proteobacteria Subgroup 2.” “Gram-negative Proteobacteria Subgroup 2” is defined as the group of Proteobacteria of the following genera (with the total numbers of catalog-listed, publicly-available, deposited strains thereof indicated in parenthesis, all deposited at ATCC, except as otherwise indicated): Acidomonas (2); Acetobacter (93); Gluconobacter (37); Brevundimonas (23); Beyerinckia (13); Derxia (2); Brucella (4); Agrobacterium (79); Chelatobacter (2); Ensifer (3); Rhizobium (144); Sinorhizobium (24); Blastomonas (1); Sphingomonas (27); Alcaligenes (88); Bordetella (43); Burkholderia (73); Ralstonia (33); Acidovorax (20); Hydrogenophaga (9); Zoogloea (9); Methylobacter (2); Methylocaldum (1 at NCIMB); Methylococcus (2); Methylomicrobium (2); Methylomonas (9); Methylosarcina (1); Methylosphaera; Azomonas (9); Azorhizophilus (5); Azotobacter (64); Cellvibrio (3); Oligella (5); Pseudomonas (1139); Francisella (4); Xanthomonas (229); Stenotrophomonas (50); and Oceanimonas (4).

Exemplary host cell species of “Gram-negative Proteobacteria Subgroup 2” include, but are not limited to the following bacteria (with the ATCC or other deposit numbers of exemplary strain(s) thereof shown in parenthesis): Acidomonas methanolica (ATCC 43581); Acetobacter aceti (ATCC 15973); Gluconobacter oxydans (ATCC 19357); Brevundimonas diminuta (ATCC 11568); Beijerinckia indica (ATCC 9039 and ATCC 19361); Derxia gummosa (ATCC 15994); Brucella melitensis (ATCC 23456), Brucella abortus (ATCC 23448); Agrobacterium tumefaciens (ATCC 23308), Agrobacterium radiobacter (ATCC 19358), Agrobacterium rhizogenes (ATCC 11325); Chelatobacter heintzii (ATCC 29600); Ensifer adhaerens (ATCC 33212); Rhizobium leguminosarum (ATCC 10004); Sinorhizobium fredii (ATCC 35423); Blastomonas natatoria (ATCC 35951); Sphingomonas paucimobilis (ATCC 29837); Alcaligenes faecalis (ATCC 8750); Bordetella pertussis (ATCC 9797); Burkholderia cepacia (ATCC 25416); Ralstonia pickettii (ATCC 27511); Acidovorax facilis (ATCC 11228); Hydrogenophagaflava (ATCC 33667); Zoogloea ramigera (ATCC 19544); Methylobacter luteus (ATCC 49878); Methylocaldum gracile (NCIMB 11912); Methylococcus capsulatus (ATCC 19069); Methylomicrobium agile (ATCC 35068); Methylomonas methanica (ATCC 35067); Methylosarcina fibrata (ATCC 700909); Methylosphaera hansonii (ACAM 549); Azomonas agilis (ATCC 7494); Azorhizophilus paspali (ATCC 23833); Azotobacter chroococcum (ATCC 9043); Cellvibrio mixtus (UQM 2601); Oligella urethralis (ATCC 17960); Pseudomonas aeruginosa (ATCC 10145), Pseudomonas fluorescens (ATCC 35858); Francisella tularensis (ATCC 6223); Stenotrophomonas maltophilia (ATCC 13637); Xanthomonas campestris (ATCC 33913); and Oceanimonas doudoroffii (ATCC 27123).

In another embodiment, the host cell is selected from “Gram-negative Proteobacteria Subgroup 3.” “Gram-negative Proteobacteria Subgroup 3” is defined as the group of Proteobacteria of the following genera: Brevundimonas; Agrobacterium; Rhizobium; Sinorhizobium; Blastomonas; Sphingomonas; Alcaligenes; Burkholderia; Ralstonia; Acidovorax; Hydrogenophaga; Methylobacter; Methylocaldum; Methylococcus; Methylomicrobium; Methylomonas; Methylosarcina; Methylosphaera; Azomonas; Azorhizophilus; Azotobacter; Cellvibrio; Oligella; Pseudomonas; Teredinibacter; Francisella; Stenotrophomonas; Xanthomonas; and Oceanimonas.

In another embodiment, the host cell is selected from “Gram-negative Proteobacteria Subgroup 4.” “Gram-negative Proteobacteria Subgroup 4” is defined as the group of Proteobacteria of the following genera: Brevundimonas; Blastomonas; Sphingomonas; Burkholderia; Ralstonia; Acidovorax; Hydrogenophaga; Methylobacter; Methylocaldum; Methylococcus; Methylomicrobium; Methylomonas; Methylosarcina; Methylosphaera; Azomonas; Azorhizophilus; Azotobacter; Cellvibrio; Oligella; Pseudomonas; Teredinibacter; Francisella; Stenotrophomonas; Xanthomonas; and Oceanimonas.

In another embodiment, the host cell is selected from “Gram-negative Proteobacteria Subgroup 5.” “Gram-negative Proteobacteria Subgroup 5” is defined as the group of Proteobacteria of the following genera: Methylobacter; Methylocaldum; Methylococcus; Methylomicrobium; Methylomonas; Methylosarcina; Methylosphaera; Azomonas; Azorhizophilus; Azotobacter; Cellvibrio; Oligella; Pseudomonas; Teredinibacter; Francisella; Stenotrophomonas; Xanthomonas; and Oceanimonas.

The host cell can be selected from “Gram-negative Proteobacteria Subgroup 6.” “Gram-negative Proteobacteria Subgroup 6” is defined as the group of Proteobacteria of the following genera: Brevundimonas; Blastomonas; Sphingomonas; Burkholderia; Ralstonia; Acidovorax; Hydrogenophaga; Azomonas; Azorhizophilus; Azotobacter; Cellvibrio; Oligella; Pseudomonas; Teredinibacter; Stenotrophomonas; Xanthomonas; and Oceanimonas.

The host cell can be selected from “Gram-negative Proteobacteria Subgroup 7.” “Gram-negative Proteobacteria Subgroup 7” is defined as the group of Proteobacteria of the following genera: Azomonas; Azorhizophilus; Azotobacter; Cellvibrio; Oligella; Pseudomonas; Teredinibacter; Stenotrophomonas; Xanthomonas; and Oceanimonas.

The host cell can be selected from “Gram-negative Proteobacteria Subgroup 8.” “Gram-negative Proteobacteria Subgroup 8” is defined as the group of Proteobacteria of the following genera: Brevundimonas; Blastomonas; Sphingomonas; Burkholderia; Ralstonia; Acidovorax; Hydrogenophaga; Pseudomonas; Stenotrophomonas; Xanthomonas; and Oceanimonas.

The host cell can be selected from “Gram-negative Proteobacteria Subgroup 9.” “Gram-negative Proteobacteria Subgroup 9” is defined as the group of Proteobacteria of the following genera: Brevundimonas; Burkholderia; Ralstonia; Acidovorax; Hydrogenophaga; Pseudomonas; Stenotrophomonas; and Oceanimonas.

The host cell can be selected from “Gram-negative Proteobacteria Subgroup 10.” “Gram-negative Proteobacteria Subgroup 10” is defined as the group of Proteobacteria of the following genera: Burkholderia; Ralstonia; Pseudomonas; Stenotrophomonas; and Xanthomonas.

The host cell can be selected from “Gram-negative Proteobacteria Subgroup 11.” “Gram-negative Proteobacteria Subgroup 11” is defined as the group of Proteobacteria of the genera: Pseudomonas; Stenotrophomonas; and Xanthomonas. The host cell can be selected from “Gram-negative Proteobacteria Subgroup 12.” “Gram-negative Proteobacteria Subgroup 12” is defined as the group of Proteobacteria of the following genera: Burkholderia; Ralstonia; Pseudomonas. The host cell can be selected from “Gram-negative Proteobacteria Subgroup 13.” “Gram-negative Proteobacteria Subgroup 13” is defined as the group of Proteobacteria of the following genera: Burkholderia; Ralstonia; Pseudomonas; and Xanthomonas. The host cell can be selected from “Gram-negative Proteobacteria Subgroup 14.” “Gram-negative Proteobacteria Subgroup 14” is defined as the group of Proteobacteria of the following genera: Pseudomonas and Xanthomonas. The host cell can be selected from “Gram-negative Proteobacteria Subgroup 15.” “Gram-negative Proteobacteria Subgroup 15” is defined as the group of Proteobacteria of the genus Pseudomonas.

The host cell can be selected from “Gram-negative Proteobacteria Subgroup 16.” “Gram-negative Proteobacteria Subgroup 16” is defined as the group of Proteobacteria of the following Pseudomonas species (with the ATCC or other deposit numbers of exemplary strain(s) shown in parenthesis): Pseudomonas abietaniphila (ATCC 700689); Pseudomonas aeruginosa (ATCC 10145); Pseudomonas alcaligenes (ATCC 14909); Pseudomonas anguilliseptica (ATCC 33660); Pseudomonas citronellolis (ATCC 13674); Pseudomonas flavescens (ATCC 51555); Pseudomonas mendocina (ATCC 25411); Pseudomonas nitroreducens (ATCC 33634); Pseudomonas oleovorans (ATCC 8062); Pseudomonas pseudoalcaligenes (ATCC 17440); Pseudomonas resinovorans (ATCC 14235); Pseudomonas straminea (ATCC 33636); Pseudomonas agarici (ATCC 25941); Pseudomonas alcaliphila; Pseudomonas alginovora; Pseudomonas andersonii; Pseudomonas asplenii (ATCC 23835); Pseudomonas azelaica (ATCC 27162); Pseudomonas beyerinckii (ATCC 19372); Pseudomonas borealis; Pseudomonas boreopolis (ATCC 33662); Pseudomonas brassicacearum; Pseudomonas butanovora (ATCC 43655); Pseudomonas cellulosa (ATCC 55703); Pseudomonas aurantiaca (ATCC 33663); Pseudomonas chlororaphis (ATCC 9446, ATCC 13985, ATCC 17418, ATCC 17461); Pseudomonas fragi (ATCC 4973); Pseudomonas lundensis (ATCC 49968); Pseudomonas taetrolens (ATCC 4683); Pseudomonas cissicola (ATCC 33616); Pseudomonas coronafaciens; Pseudomonas diterpeniphila; Pseudomonas elongata (ATCC 10144); Pseudomonasflectens (ATCC 12775); Pseudomonas azotoformans; Pseudomonas brenneri; Pseudomonas cedrella; Pseudomonas corrugata (ATCC 29736); Pseudomonas extremorientalis; Pseudomonas fluorescens (ATCC 35858); Pseudomonas gessardii; Pseudomonas libanensis; Pseudomonas mandelii (ATCC 700871); Pseudomonas marginalis (ATCC 10844); Pseudomonas migulae; Pseudomonas mucidolens (ATCC 4685); Pseudomonas orientalis; Pseudomonas rhodesiae; Pseudomonas synxantha (ATCC 9890); Pseudomonas tolaasii (ATCC 33618); Pseudomonas veronii (ATCC 700474); Pseudomonas frederiksbergensis; Pseudomonas geniculata (ATCC 19374); Pseudomonas gingeri; Pseudomonas graminis; Pseudomonas grimontii; Pseudomonas halodenitrificans; Pseudomonas halophila; Pseudomonas hibiscicola (ATCC 19867); Pseudomonas huttiensis (ATCC 14670); Pseudomonas hydrogenovora; Pseudomonas jessenii (ATCC 700870); Pseudomonas kilonensis; Pseudomonas lanceolata (ATCC 14669); Pseudomonas lini; Pseudomonas marginate (ATCC 25417); Pseudomonas mephitica (ATCC 33665); Pseudomonas denitrificans (ATCC 19244); Pseudomonas pertucinogena (ATCC 190); Pseudomonas pictorum (ATCC 23328); Pseudomonas psychrophila; Pseudomonas filva (ATCC 31418); Pseudomonas monteilii (ATCC 700476); Pseudomonas mosselii; Pseudomonas oryzihabitans (ATCC 43272); Pseudomonas plecoglossicida (ATCC 700383); Pseudomonas putida (ATCC 12633); Pseudomonas reactans; Pseudomonas spinosa (ATCC 14606); Pseudomonas balearica; Pseudomonas luteola (ATCC 43273); Pseudomonas stutzeri (ATCC 17588); Pseudomonas amygdali (ATCC 33614); Pseudomonas avellanae (ATCC 700331); Pseudomonas caricapapayae (ATCC 33615); Pseudomonas cichorii (ATCC 10857); Pseudomonas ficuserectae (ATCC 35104); Pseudomonas fuscovaginae; Pseudomonas meliae (ATCC 33050); Pseudomonas syringae (ATCC 19310); Pseudomonas viridiflava (ATCC 13223); Pseudomonas thermocarboxydovorans (ATCC 35961); Pseudomonas thermotolerans; Pseudomonas thivervalensis; Pseudomonas vancouverensis (ATCC 700688); Pseudomonas wisconsinensis; and Pseudomonas xiamenensis.

The host cell can be selected from “Gram-negative Proteobacteria Subgroup 17.” “Gram-negative Proteobacteria Subgroup 17” is defined as the group of Proteobacteria known in the art as the “fluorescent Pseudomonads” including those belonging, e.g., to the following Pseudomonas species: Pseudomonas azotoformans; Pseudomonas brenneri; Pseudomonas cedrella; Pseudomonas corrugata; Pseudomonas extremorientalis; Pseudomonas fluorescens; Pseudomonas gessardii; Pseudomonas libanensis; Pseudomonas mandelii; Pseudomonas marginalis; Pseudomonas migulae; Pseudomonas mucidolens; Pseudomonas orientalis; Pseudomonas rhodesiae; Pseudomonas synxantha; Pseudomonas tolaasii; and Pseudomonas veronii.

In one of these embodiments, the host cell can be selected from “Gram-negative Proteobacteria Subgroup 18.” “Gram-negative Proteobacteria Subgroup 18” is defined as the group of all subspecies, varieties, strains, and other sub-special units of the species Pseudomonas fluorescens, including those belonging, e.g., to the following (with the ATCC or other deposit numbers of exemplary strain(s) shown in parenthesis): Pseudomonas fluorescens biotype A, also called biovar 1 or biovar I (ATCC 13525); Pseudomonas fluorescens biotype B, also called biovar 2 or biovar II (ATCC 17816); Pseudomonas fluorescens biotype C, also called biovar 3 or biovar III (ATCC 17400); Pseudomonas fluorescens biotype F, also called biovar 4 or biovar IV (ATCC 12983); Pseudomonas fluorescens biotype G, also called biovar 5 or biovar V (ATCC 17518); Pseudomonas fluorescens biovar VI; Pseudomonas fluorescens Pf0-1; Pseudomonas fluorescens Pf-5 (ATCC BAA-477); Pseudomonas fluorescens SBW25; and Pseudomonas fluorescens subsp. cellulosa (NCIMB 10462).

The host cell can be selected from “Gram-negative Proteobacteria Subgroup 19.” “Gram-negative Proteobacteria Subgroup 19” is defined as the group of all strains of Pseudomonas fluorescens biotype A. A particularly preferred strain of this biotype is P. fluorescens strain MB101 (see U.S. Pat. No. 5,169,760 to Wilcox), and derivatives thereof. An example of a preferred derivative thereof is P. fluorescens strain MB214, constructed by inserting into the MB101 chromosomal asd (aspartate dehydrogenase gene) locus, a native E. coli PlacI-lacI-lacZYA construct (i.e. in which PlacZ was deleted).

Additional P. fluorescens strains that can be used in the present invention include Pseudomonas fluorescens Migula and Pseudomonas fluorescens Loitokitok, having the following ATCC designations: [NCIB 8286]; NRRL B-1244; NCIB 8865 strain CO1; NCIB 8866 strain CO₂; 1291 [ATCC 17458; IFO 15837; NCIB 8917; LA; NRRL B-1864; pyrrolidine; PW2 [ICMP 3966; NCPPB 967; NRRL B-899]; 13475; NCTC 10038; NRRL B-1603 [6; IFO 15840]; 52-1C; CCEB 488-A [BU 140]; CCEB 553 [EM 15/47]; IAM 1008 [AHH-27]; IAM 1055 [AHH-23]; 1 [IFO 15842]; 12 [ATCC 25323; NIH 11; den Dooren de Jong 216]; 18 [IFO 15833; WRRL P-7]; 93 [TR-10]; 108 [52-22; IFO 15832]; 143 [IFO 15836; PL]; 149 [2-40-40; IFO 15838]; 182 [IFO 3081; PJ 73]; 184 [IFO 15830]; 185 [W2 L-1]; 186 [IFO 15829; PJ 79]; 187 [NCPPB 263]; 188 [NCPPB 316]; 189 [PJ227; 1208]; 191 [IFO 15834; PJ 236; 22/1]; 194 [Klinge R-60; PJ 253]; 196 [PJ 288]; 197 [PJ 290]; 198 [PJ 302]; 201 [PJ 368]; 202 [PJ 372]; 203 [PJ 376]; 204 [IFO 15835; PJ 682]; 205 [PJ 686]; 206 [PJ 692]; 207 [PJ 693]; 208 [PJ 722]; 212. [PJ 832]; 215 [PJ 849]; 216 [PJ 885]; 267 [B-9]; 271 [B-1612]; 401 [C71A; IFO 15831; PJ 187]; NRRL B-3178 [4; IFO. 15841]; KY 8521; 3081; 30-21; [IFO 3081]; N; PYR; PW; D946-B83 [BU 2183; FERM-P 3328]; P-2563 [FERM-P 2894; IFO 13658]; IAM-1126 [43F]; M-1; A506 [A5-06]; A505 [A5-05-1]; A526 [A5-26]; B69; 72; NRRL B-4290; PMW6 [NCIB 11615]; SC 12936; Al [IFO 15839]; F 1847 [CDC-EB]; F 1848 [CDC 93]; NCIB 10586; P17; F-12; AmMS 257; PRA25; 6133D02; 6519E01; Ni; SC15208; BNL-WVC; NCTC 2583 [NCIB 8194]; H13; 1013 [ATCC 11251; CCEB 295]; IFO 3903; 1062; or Pf-5.

Other suitable hosts include those classified in other parts of the reference, such as Gram (+) Proteobacteria. In one embodiment, the host cell is an E. coli. The genome sequence for E. coli has been established for E. coli MG1655 (Blattner, et al. (1997) The complete genome sequence of Escherichia coli K-12, Science 277(5331): 1453-74) and DNA microarrays are available commercially for E. coli K12 (MWG Inc, High Point, N.C.). E. coli can be cultured in either a rich medium such as Luria-Bertani (LB) (10 g/L tryptone, 5 g/L NaCl, 5 g/L yeast extract) or a defined minimal medium such as M9 (6 g/L Na₂HPO₄, 3 g/L KH₂PO₄, 1 g/L NH₄C1, 0.5 g/L NaCl, pH 7.4) with an appropriate carbon source such as 1% glucose. Routinely, an over night culture of E. coli cells is diluted and inoculated into fresh rich or minimal medium in either a shake flask or a fermentor and grown at 37° C.

A host can also be of mammalian origin, such as a cell derived from a mammal including any human or non-human mammal. Mammals can include, but are not limited to primates, monkeys, porcine, ovine, bovine, rodents, ungulates, pigs, swine, sheep, lambs, goats, cattle, deer, mules, horses, monkeys, apes, dogs, cats, rats, and mice.

A host cell may also be of plant origin. Any plant can be selected for the identification of genes and regulatory elements. Examples of suitable plant targets for the isolation of genes and regulatory elements would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassaya, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radiscchio, radish, rapeseed, raspberry, rice, rye, sorghum, Southern pine, soybean, spinach, squash, strawberry, sugarbeet, sugarcane, sunflower, sweet potato, sweetgum, tangerine, tea, tobacco, tomato, triticale, turf, turnip, a vine, watermelon, wheat, yams, and zucchini In some embodiments, plants useful in the method are Arabidopsis, corn, wheat, soybean, and cotton.

2. Cell Growth Conditions

The cell growth conditions for the host cells described herein can include that which facilitates replication of the expression vectors or expression constructs described herein, expression of the protein of interest from the plasmid, and/or that which facilitates fermentation of the expressed protein of interest. For the identification of a population of expression constructs optimal for the expression of a heterologous protein of interest, the population of expression constructs is introduced into a population of host cells of interest, and the host cells are grown under conditions sufficient for the expression and/or secretion of the polypeptide in at least one host cell.

As used herein, the term “fermentation” includes both embodiments in which literal fermentation is employed and embodiments in which other, non-fermentative culture modes are employed. Fermentation may be performed at any scale. In one embodiment, the fermentation medium may be selected from among rich media, minimal media, and mineral salts media; a rich medium may be used, but is preferably avoided. In another embodiment either a minimal medium or a mineral salts medium is selected. In still another embodiment, a minimal medium is selected. In yet another embodiment, a mineral salts medium is selected. Mineral salts media are particularly preferred.

Mineral salts media consists of mineral salts and a carbon source such as, e.g., glucose, sucrose, or glycerol. Examples of mineral salts media include, e.g., M9 medium, Pseudomonas medium (ATCC 179), Davis and Mingioli medium (see, BD Davis & ES Mingioli (1950) in J. Bact. 60:17-28). The mineral salts used to make mineral salts media include those selected from among, e.g., potassium phosphates, ammonium sulfate or chloride, magnesium sulfate or chloride, and trace minerals such as calcium chloride, borate, and sulfates of iron, copper, manganese, and zinc. The mineral salts medium does not have, but can include an organic nitrogen source, such as peptone, tryptone, amino acids, or a yeast extract. An inorganic nitrogen source can also be used and selected from among, e.g., ammonium salts, aqueous ammonia, and gaseous ammonia. In comparison to mineral salts media, minimal media can also contain mineral salts and a carbon source, but can be supplemented with, e.g., low levels of amino acids, vitamins, peptones, or other ingredients, though these are added at very minimal levels.

The expression system according to the present invention can be cultured in any fermentation format. For example, batch, fed-batch, semi-continuous, and continuous fermentation modes may be employed herein. Wherein the protein is excreted into the extracellular medium, continuous fermentation is preferred.

The expression systems according to the present invention are useful for protein expression at any scale (i.e. volume) of fermentation. Thus, e.g., microliter-scale, centiliter scale, and deciliter scale fermentation volumes may be used; and 1 Liter scale and larger fermentation volumes can be used. In one embodiment, the fermentation volume will be at or above 1 Liter. In another embodiment, the fermentation volume will be at or above 5 Liters, 10 Liters, 15 Liters, 20 Liters, 25 Liters, 50 Liters, 75 Liters, 100 Liters, 200 Liters, 500 Liters, 1,000 Liters, 2,000 Liters, 5,000 Liters, 10,000 Liters or 50,000 Liters.

In the present invention, growth, culturing, and/or fermentation of the transformed host cells is performed within a temperature range permitting survival of the host cells, preferably a temperature within the range of about 4° C. to about 55° C., inclusive. Thus, e.g., the terms “growth” (and “grow,” “growing”), “culturing” (and “culture”), and “fermentation” (and “ferment,” “fermenting”), as used herein in regard to the host cells of the present invention, inherently means “growth,” “culturing,” and “fermentation,” within a temperature range of about 4° C. to about 55° C., inclusive. In addition, “growth” is used to indicate both biological states of active cell division and/or enlargement, as well as biological states in which a non-dividing and/or non-enlarging cell is being metabolically sustained, the latter use of the term “growth” being synonymous with the term “maintenance.”

In some embodiments, the expression system comprises a Pseudomonas host cell, e.g. Psuedomonas fluorescens. An advantage in using Pseudomonas fluorescens in expressing heterologous proteins includes the ability of Pseudomonas fluorescens to be grown in high cell densities compared to E. coli or other bacterial expression systems. To this end, Pseudomonas fluorescens expressions systems according to the present invention can provide a cell density of about 20 g/L or more. The Pseudomonas fluorescens expressions systems according to the present invention can likewise provide a cell density of at least about 70 g/L, as stated in terms of biomass per volume, the biomass being measured as dry cell weight.

In one embodiment, the cell density will be at least about 20 g/L. In another embodiment, the cell density will be at least about 25 g/L, about 30 g/L, about 35 g/L, about 40 g/L, about 45 g/L, about 50 g/L, about 60 g/L, about 70 g/L, about 80 g/L, about 90 g/L., about 100 g/L, about 110 g/L, about 120 g/L, about 130 g/L, about 140 g/L, about or at least about 150 g/L.

In another embodiments, the cell density at induction will be between about 20 g/L and about 150 g/L; between about 20 g/L and about 120 g/L; about 20 g/L and about 80 g/L; about 25 g/L and about 80 g/L; about 30 g/L and about 80 g/L; about 35 g/L and about 80 g/L; about 40 g/L and about 80 g/L; about 45 g/L and about 80 g/L; about 50 g/L and about 80 g/L; about 50 g/L and about 75 g/L; about 50 g/L and about 70 g/L; about 40 g/L and about 80 g/L.

3. Expression of the Polypeptide of Interest

Methods of the invention comprise culturing host cells comprising an expression construct under conditions that allow for the expression of the polypeptide of interest. Those host cells that express sufficient levels of the polypeptide of interest are then selected and the expression construct is isolated from the selected host cell. As discussed elsewhere herein, a “sufficient level” is intended to describe the quality (e.g., activity, solubility, processing, etc.) and/or quantity (e.g., level of total protein produced and/or secreted) of the polypeptide of interest. Individual host cell populations comprising genotypically distinct expression constructs can be distinguished, for example, by growing isolated colonies of each population of host cell and individually expanding each population in independent cultures.

A sufficient level of protein expression can be described in terms of the levels of properly processed polypeptide per gram of protein produced, or per gram of host protein. The level of recoverable protein or polypeptide produced per gram of recombinant or per gram of host cell protein can also be measured. The expression level of a polypeptide of interest can also refer to a combination of the level of total protein, the level of properly processed protein, or the level of active or soluble protein.

The expression of a polypeptide of interest can also refer to the solubility of the polypeptide. The polypeptide of interest can be produced and recovered from the cytoplasm, periplasm or extracellular medium of the host cell. The polypeptide can be insoluble or soluble. The polypeptide can include one or more targeting sequences or sequences to assist purification, as discussed supra.

The term “soluble” as used herein means that the protein is not precipitated by centrifugation at between approximately 5,000 and 20,000× gravity when spun for 10-30 minutes in a buffer under physiological conditions. Soluble proteins are not part of an inclusion body or other precipitated mass. Similarly, “insoluble” means that the protein or polypeptide can be precipitated by centrifugation at between 5,000 and 20,000× gravity when spun for 10-30 minutes in a buffer under physiological conditions. Insoluble proteins or polypeptides can be part of an inclusion body or other precipitated mass. The term “inclusion body” is meant to include any intracellular body contained within a cell wherein an aggregate of proteins or polypeptides has been sequestered.

In those embodiments in which the polypeptide of interest has been targeted to the periplasm of the host cell, an expression construct that results in a “sufficient level of expression” refers to a construct that results in the accumulation of at least 0.1 g/L protein in the periplasmic compartment. In another embodiment, the construct results in the production of about 0.1 to about 10 g/L periplasmic protein in the cell, or at least about 0.2, about 0.3, about 0.4, about 0.5, about 0.6, about 0.7, about 0.8, about 0.9 or at least about 1.0 g/L periplasmic protein. In one embodiment, the total protein or polypeptide of interest produced is at least 1.0 g/L, at least about 2 g/L, at least about 3 g/L, about 4 g/L, about 5 g/L, about 6 g/L, about 7 g/L, about 8 g/L, about 10 g/L, about 15 g/L, about 20 g/L, at least about 25 g/L, or greater. In some embodiments, the amount of periplasmic protein produced is at least about 5%, about 10%, about 15%, about 20%, about 25%, about 30%, about 40%, about 50%, about 60%, about 70%, about 80%, about 90%, about 95%, about 96%, about 97%, about 98%, about 99%, or more of total protein or polypeptide of interest produced.

In practice, heterologous proteins targeted to the periplasm are often found in the broth (see European Patent No. EP 0 288 451), possibly because of damage to or an increase in the fluidity of the outer cell membrane. The rate of this “passive” secretion may be increased by using a variety of mechanisms that permeabilize the outer cell membrane: colicin (Miksch et al. (1997) Arch. Microbiol. 167: 143-150); growth rate (Shokri et al. (2002) App Miocrobiol Biotechnol 58:386-392); TolIII overexpression (Wan and Baneyx (1998) Protein Expression Purif. 14: 13-22); bacteriocin release protein (Hsiung et al. (1989) Bio/Technology 7: 267-71), colicin A lysis protein (Lloubes et al. (1993) Biochimie 75: 451-8) mutants that leak periplasmic proteins (Furlong and Sundstrom (1989) Developments in Indus. Microbio. 30: 141-8); fusion partners (Jeong and Lee (2002) Appl. Environ. Microbio. 68: 4979-4985); recovery by osmotic shock (Taguchi et al. (1990) Biochimica Biophysica Acta 1049: 278-85). Transport of engineered proteins to the periplasmic space with subsequent localization in the broth has been used to produce properly folded and active proteins in E. coli (Wan and Baneyx (1998) Protein Expression Purif. 14: 13-22; Simmons et al. (2002) J. Immun. Meth. 263: 133-147; Lundell et al. (1990) J. Indust. Microbio. 5: 215-27).

In one embodiment, the construct results in the production of at least 0.1 g/L correctly processed protein. A correctly processed protein has an amino terminus of the native protein. In another embodiment, the method produces 0.1 to 10 g/L correctly processed protein in the cell, including at least about 0.2, about 0.3, about 0.4, about 0.5, about 0.6, about 0.7, about 0.8, about 0.9 or at least about 1.0 g/L correctly processed protein. In another embodiment, the total correctly processed protein or polypeptide of interest produced is at least 1.0 g/L, at least about 2 g/L, at least about 3 g/L, about 4 g/L, about 5 g/L, about 6 g/L, about 7 g/L, about 8 g/L, about 10 g/L, about 15 g/L, about 20 g/L, about 25 g/L, about 30 g/L, about 35 g/l, about 40 g/l, about 45 g/l, at least about 50 g/L, or greater. In some embodiments, the amount of correctly processed protein produced is at least about 5%, about 10%, about 15%, about 20%, about 25%, about 30%, about 40%, about 50%, about 60%, about 70%, about 80%, about 90%, about 95%, about 96%, about 97%, about 98%, at least about 99%, or more of total recombinant protein in a correctly processed form.

In some embodiments, host cells comprising expression constructs sufficient for the expression of a polypeptide of interest express the polypeptide of interest at least about 5%, at least about 10%, about 15%, about 20%, about 25%, about 30%, about 40%, about 45%, about 50%, about 55%, about 60%, about 65%, about 70%, about 75%, or greater of total cell protein (tcp). “Percent total cell protein” is the amount of protein or polypeptide in the host cell as a percentage of aggregate cellular protein. The determination of the percent total cell protein is well known in the art.

In a particular embodiment, the selected host cell can have a polypeptide expression level of at least 1% tcp and a cell density of at least 40 g/L, when grown (i.e. within a temperature range of about 4° C. to about 55° C., including about 10° C., about 15° C., about 20° C., about 25° C., about 30° C., about 35° C., about 40° C., about 45° C., and about 50° C.) in a mineral salts medium. In a particularly preferred embodiment, the selected host cell will have a protein or polypeptide expression level of at least 5% tcp and a cell density of at least 40 g/L, when grown (i.e. within a temperature range of about 4° C. to about 55° C., inclusive) in a mineral salts medium at a fermentation scale of at least about 10 Liters.

In some embodiments, the method may also include the step of purifying the protein or polypeptide of interest from the periplasm, from the extracellular media, or from a cellular lysate by any method known to one of ordinary skill in the art.

In some embodiments, host cells that comprise an expression construct sufficient for the expression of a polypeptide of interest are those that produce the polypeptide of interest with a certain level of activity. The term “active” means the presence of biological activity, wherein the biological activity is comparable or substantially corresponds to the biological activity of a corresponding native polypeptide. In the context of polypeptides, this typically means that a polynucleotide or polypeptide comprises a biological function or effect that has at least about 20%, about 50%, at least about 60-80%, at least about 90-95%, at least about 100%, at least about 110%, at least about 120%, at least about 130%, at least about 140%, at least about 150%, at least about 175%, at least about 2-fold, at least about 3-fold, at least about 4-fold or greater activity compared to the corresponding native polypeptide using standard parameters. The determination of polypeptide activity can be performed utilizing corresponding standard, targeted comparative biological assays for particular polypeptides. One indication that a polypeptide of interest maintains biological activity is that the polypeptide is immunologically cross reactive with the native polypeptide.

Active polypeptides can have a specific activity of at least about 20%, at least about 30%, at least about 40%, about 50%, about 60%, at least about 70%, about 80%, about 90%, or at least about 95% that of the native polypeptide that the sequence is derived from. Further, in those embodiments in which the polypeptide of interest is an enzyme, the substrate specificity (k_{cat}/Kₘ) is optionally substantially similar to the native polypeptide. Typically, k_{cat}/Kₘ will be at least about 30%, about 40%, about 50%, about 60%, about 70%, about 80%, at least about 90%, at least about 95%, or greater. Methods of assaying and quantifying measures of polypeptide activity and substrate specificity (k_{cat}/Kₘ), are well known to those of skill in the art.

The activity of the polypeptide of interest can be also compared with a previously established native polypeptide standard activity. Alternatively, the activity of the polypeptide of interest can be determined in a simultaneous, or substantially simultaneous, comparative assay with the native polypeptide. For example, in vitro assays can be used to determine any detectable interaction between a polypeptide of interest and a target, e.g. between an expressed enzyme and substrate, between expressed hormone and hormone receptor, between expressed antibody and antigen, etc. Such detection can include the measurement of calorimetric changes, proliferation changes, cell death, cell repelling, changes in radioactivity, changes in solubility, changes in molecular weight as measured by gel electrophoresis and/or gel exclusion methods, phosphorylation abilities, antibody specificity assays such as ELISA assays, etc. In addition, in vivo assays include, but are not limited to, assays to detect physiological effects of the produced protein or polypeptide in comparison to physiological effects of the native polypeptide, e.g. weight gain, change in electrolyte balance, change in blood clotting time, changes in clot dissolution and the induction of antigenic response. Generally, any in vitro or in vivo assay can be used to determine the active nature of the polypeptide of interest that allows for a comparative analysis to the native polypeptide so long as such activity is assayable. Alternatively, the polypeptides produced in the present invention can be assayed for the ability to stimulate or inhibit interaction between the polypeptide and a molecule that normally interacts with the polypeptide, e.g. a substrate or a component of the signal pathway with which the native protein normally interacts. Such assays can typically include the steps of combining the polypeptide with a substrate molecule under conditions that allow the polypeptide to interact with the target molecule, and detect the biochemical consequence of the interaction with the polypeptide and the target molecule.

Assays that can be utilized to determine polypeptide activity are described, for example, in Ralph, P. J., et al. (1984) J. Immunol. 132:1858 or Saiki et al. (1981) J. Immunol. 127:1044, Steward, W. E. II (1980) The Interferon Systems. Springer-Verlag, Vienna and New York, Broxmeyer, H. E., et al. (1982) Blood 60:595, Molecular Cloning: A Laboratory Manua”, 2d ed., Cold Spring Harbor Laboratory Press, Sambrook, J., E. F. Fritsch and T. Maniatis eds., 1989, and Methods in Enzymology: Guide to Molecular Cloning Techniques, Academic Press, Berger, S. L. and A. R. Kimmel eds., 1987, A K Patra et al., Protein Expr Purif, 18(2): p/182-92 (2000), Kodama et al., J. Biochem. 99: 1465-1472 (1986); Stewart et al., Proc. Nat'l Acad. Sci. USA 90: 5209-5213 (1993); (Lombillo et al., J. Cell Biol. 128:107-115 (1995); (Vale et al., Cell 42:39-50 (1985).

One or more host cell(s) comprising an expression construct sufficient for the expression of a polypeptide of interest can be selected using any of the criteria discussed above, or any other suitable criteria relevant to the particular protein(s) being expressed. As discussed elsewhere herein, the threshold value for selection depends on the nature of the polypeptide of interest, as well as the intended use of the polypeptide.

Once a host cell that expresses the polypeptide of interest at a sufficient level has been selected, the expression construct can be isolated through the use of any well-known method or commercially available kit for vector purification. The sequence identity of the region of the construct that comprises the regulatory elements and/or polynucleotide encoding the polypeptide of interest can be determined through any DNA sequencing procedure known to one of ordinary skill in the art.

The following examples are offered by way of illustration and not by way of limitation.

### EXPERIMENTAL

### Example 1

### Cloning of the phoA Gene into an Expression Vector with the pbp Secretion Leader

The phoA gene was subcloned into an expression vector containing regulatory sequences, including the phosphate binding protein (pbp) secretion leader sequence.

### Modification of Expression Vector

The pDOW1169 expression vector, described in Schneider et al. (2005) Biotechnology Progress 21:343-348, herein incorporated by reference, was modified to remove the SapI restriction enzyme recognition site. Specifically, the expression vector was modified by PCR, amplifying a region of pDOW1169 from a PstI site to a BsaAI site with the oligonucleotide primers comprised of the sequence set forth in SEQ ID NO:1 and SEQ ID NO:2. All PCR reactions were performed with KOD polymerase (Novagen, cat. no. 71086), according to the manufacturer's instructions. The PCR amplified synthesis product and the pDOW1169 vector were restriction digested and ligated together such that the PCR amplified fragment replaced the corresponding region in the pDOW1169 vector. The reaction effectively removed the following sequence from the parent pDOW1169 expression vector comprising the SapI restriction enzyme recognition site: GACGAGAAGAG (the SapI site is shown in bold). The resulting plasmid was named pDOW3818.

Amplification of the pDOW3818 Expression Vector with Primers Comprising Regulatory Sequences

A PCR amplification reaction was performed, wherein the pDOW3818 expression vector was amplified using oligonucleotide primers comprised of the sequences set forth in SEQ ID NO:3 and SEQ ID NO:4. SEQ ID NO:3 comprises sequence corresponding to (in the 5′ to 3′ direction) a SapI restriction enzyme recognition site, a pbp secretion leader sequence ending in GCC (an alanine codon), an ATG start codon, a ribosome binding site, and sequence that is complementary to the 5′ untranslated region (5′ UTR) found within pDOW3818. SEQ ID NO:4 comprises sequence corresponding to (in the 5′ to 3′ direction) a SapI restriction enzyme recognition site, stop codons in all three translational reading frames, and a sequence that is complementary to a site within the pDOW3818 sequence that is upstream of the transcription termination sequence. The PCR amplification synthesis product was restriction digested with SapI (Fermentas, cat. no. FD1934) and purified by gel extraction (Qiagen cat. no. 28704) to prepare it for ligation with the phoA gene.

Amplification of the phoA Gene

Another PCR reaction was performed using a plasmid that comprised the E. coli phoA gene as the target and the oligonucleotide primers comprising the sequences set forth in SEQ ID NO:5 and SEQ ID NO:6. SEQ ID NO:5 comprises sequence corresponding to a SapI restriction enzyme recognition site and sequence corresponding to the second codon of the mature phoA protein. SEQ ID NO:6 comprised a SapI site and the reverse complement of a stop codon. The PCR amplification synthesis product was restriction digested with SapI and purified by gel extraction as above.

TABLE 4
Oligonucleotide primer sequences

| Primer | | Sequence |
|---|---|---|
| Name | Sequence* | Listing |
| Rapid SapI | 5′ ATATCTGCAGCATACATCTGGAAGC | SEQ ID NO: 1 |
| minus F | AAAGC | |
| Rapid SapI | 5′ CGCCTGTACGTGGCCCTGAA | SEQ ID NO: 2 |
| minus R | | |
| Rapid101 | 5′ ATATGCTCTTCAGGCCACCGCGTTG | SEQ ID NO: 3 |
| | GCGGTCGCAACGCCAGCAGCGACAAAAG | |
| | TCATTGCCGCCATCAAACGTTTCAGTTT | |
| | CATAAGTTACCTCCTACTAGTAGATTAA | |
| | AATTCTGTTTCCTGTG | |
| Rapid103 | 5′ ATATGCTCTTCATAACTCGAGCCCA | SEQ ID NO: 4 |
| | AAACGAAAGG | |
| JCS541 | 5′ ATATGCTCTTCAGCCCGGACACCAG | SEQ ID NO: 5 |
| | AAATGCCTGTT | |
| JCS542-r | 5′ ATATGCTCTTCTTTACTATTATCAG | SEQ ID NO: 6 |
| | GTACCTTTCAGCCCCAGAG | |

* The SapI restriction enzyme recognition site is underlined;
the pbp signal sequence is italicized;
the ribosome binding site is italicized and underlined;
the sequences that are complementary to pDOW3818 sequences are bolded.

Ligation of pDOW3818 PCR Amplification Synthesis Product and phoA Gene PCR Amplification Synthesis Product

The PCR amplification synthesis products from the pDOW3818 and phoA gene PCR amplification reactions were ligated with a DNA ligase (NEB, cat. no. M0202, and then electroporated into the washed P. fluorescens strain DC454 before spreading the cells on a M9glucose agar plate. After colony formation, the colonies were tested for PhoA activity on BCIP plates (Chaffin, D. O. and C. E. Rubens (1998) Gene 219:91-99), the plasmid from blue colonies was purified, and the amplified region was sequenced. Sequencing showed that the phoA gene was inserted in frame into the modified pDOW3818 vector with the pbp secretion leader.

### Example 2

### Production of an Expression Construct Comprising Two Coding Regions Encoding Two Polypeptides of Interest

An expression construct is produced that comprises a polynucleotide sequence comprising two coding regions encoding two polypeptides of interest with a bidirectional transcription termination sequence disposed between the two coding regions. SapI recognition sites with non-identical overhanging ends flank both ends of the polynucleotide sequence (SapIa and SapIb). The SapIa overhanging end is complementary to a first alanine codon within a signal sequence present within the first primer. The SapIb overhanging end is complementary to a second alanine codon within a signal sequence present within the second primer. A PCR reaction is performed with the first and second primers and a vector as a template. The vector comprises two promoters and two 5′ untranslated regions (5′ UTR1 and 5′ UTR2). The first primer comprises, in the 5′ to 3′ direction, a SapIa site (comprised of a SapI recognition site and the first alanine codon of a signal sequence, a signal sequence, a ribosome binding site (RBS), and a sequence that is complementary to the 5′ UTR1 present within the vector. The second primer comprises, in the 5′ to 3′ direction, a SapIb site (comprised of a SapI recognition site and the second alanine codon of a signal sequence), a signal sequence, a RBS, and a sequence that is complementary to the 5′ UTR2 present within the vector. The expression vector produced in the PCR reaction is cleaved with SapI and ligated with the polynucleotide sequence comprising the two coding regions to form an expression construct. The orientation of the two coding regions and the regulatory sequences within the resultant expression construct are such that transcription of each coding region will proceed towards the bidirectional transcriptional termination sequence. Therefore, when the expression construct is transformed into a host cell, the host cell can be cultured in such a manner as to express the two polypeptides of interest.

### Example 3

### Identification of Pseudomonas Bidirectional Terminator

### Materials and Methods

Cloning Bidirectional Terminators Between Promoter and RBS

Linkers containing bidirectional terminators with cohesive ends for restriction site cloning were cloned into an expression vector between the promoter and ribosome binding site. For each terminator (see Table 1 for terminator sequences), a pair of complementary oligonucleotides with phosphorylated 5′ ends were designed with a SpeI overhang on one end and an XbaI overhang on the other and synthesized by Operon (04167term F & R, 02858terminator_F & R, and Tn10terminator_F & R. Each primer set was annealed as follows:

| | |
|---|---|
| 100 uM oligonucleotide 1 | 3.5 μL |
| 100 uM oligonucleotide 2 | 3.5 μL |
| 1M NaCl | 2 μL |
| 0.5M Tris.HCl(pH8) | 2 μL |
| H₂O | |

And run in the following routine:

### 97° C. for 5 min

### 65° C. for 10 min

### Cooled to 22° C. at 0.1° C./sec

The annealed primers were purified using QIAquick Nucleotide Remove kit (Qiagen cat. # 28304). The fragments were ligated into the SpeI site between the dual-operator tac promoter and ribosome binding site in plasmid pDOW1344, which had been restriction digested with SpeI followed by alkaline phosphatase treatment (CIP, NEB cat. # M0290S). Those constructs, named pDOW2942/pDOW2943 (for Term04167/76 in both orientations), pDOW2950/pDOW2951 (for Term02857/58 in both orientations), and pDOW2952/pDOW2953 (for Tn10 in both orientations), were then electroporated into the P. fluorescens host strain DC454 (ΔpyrF RX01414::lacI^{Q1}). Positive clones were confirmed by sequence analysis. The combined terminators of Term4167/76-Term2857/58 were cloned into by ligating the Term02857/58 linker into SpeI site in pDOW2943 plasmid to make pDOW2947 and pDOW2954.

DNA Sequencing

Clones were analyzed by sequencing using Big dye version 3.1 (Applied Biosystems). Reactions consisted of 2 μl of sequencing premix, 1 μl of 6.4 μM primer, 50 fmol of DNA template, 3 μL 5× buffer+H2O to adjust volume to 20 μl. Sequencing reactions were then purified using G-50 (Sigma) and loaded into the ABI3100 sequencer. Sequence data were assembled and analyzed using the Sequencer software (Gene Codes).

High Throughput (HTP) COP-GFP Expression Analysis

The P. fluorescens strains were analyzed using a standard expression protocol. Briefly, seed cultures grown in M9 medium supplemented with 1% glucose and trace elements were used to inoculate 0.5 mL of defined minimal salts medium without yeast extract (Teknova 3H1130) with 5% glycerol as the carbon source in a 2.0 mL deep 96-well plate. Following an initial growth phase at 30° C., expression via the Ptac promoter was induced with 0.3 mM isopropyl-β-D-1-thiogalactopyranoside (IPTG). Cultures were sampled by removing 10 μL of whole broth into 96 well shallow plate at I0, and at 24 hours post induction (124). Cell density was measured by optical density at 600 nm (OD600), and the relative fluorescence values were assayed using COP-GFP expression protocol with settings of Excitation 485 Emission 538, with a 530 bandpass (Schneider et al. 2004).

Construction of Plasmids Containing Bidirectional Terminators

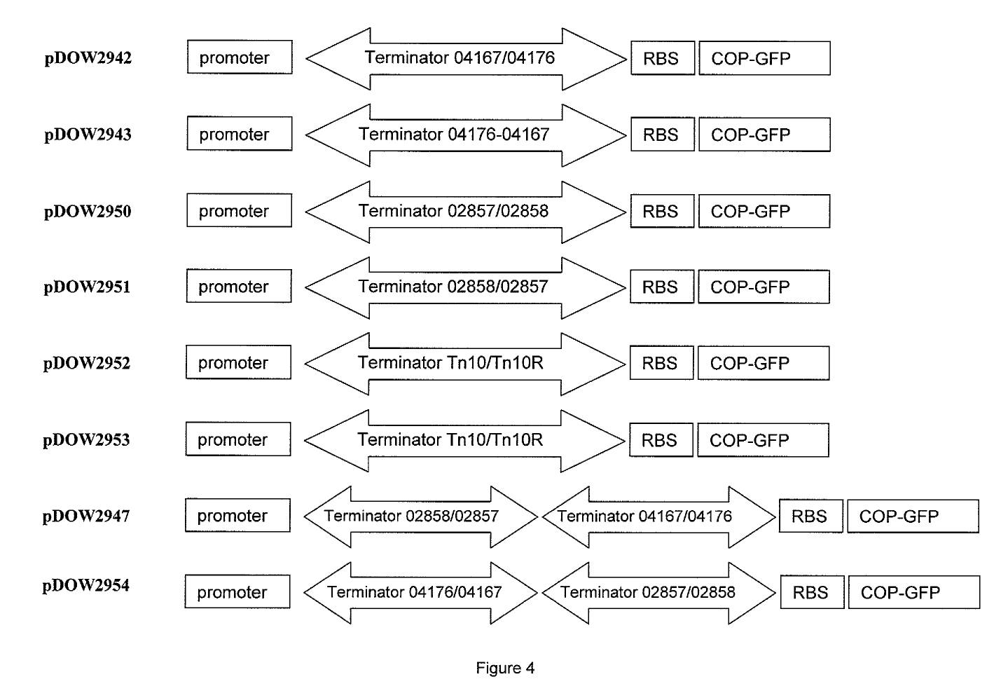

The program TransTerm (Ermolaeva et al. (2000) J Mol Biol 301(1):27-33) was used to predict putative rho-independent transcription terminators in the P. fluorescens MB101 genome. Sequences with a strong score on both strands were identified in the following Table. The putative terminators were named by using the RXF number of the closest open reading frame. The potential bidirectional terminator sequences Term4167/76 and Term02857/57 (from the P. fluorescens genome) as well as the E. coli Tn10 terminator were synthesized and cloned in both orientations in the SpeI site between the promoter and ribosome binding site in plasmid pDOW1344 containing the COP-GFP gene (FIG. 4). This resulted in two plasmids for each terminator: pDOW2942/pDOW2943 (for Term04167/76), pDOW2950/pDOW2951 (for Term02857/58), pDOW2952/pDOW2953 (for Tn10) and pDOW2947/pDOW2954 (for the combined Term04167/76-Term2857/58) (FIG. 4).

| Terminator Name | Sequence |
|---|---|
| Term04167/76 | RXF04167- |
| (SEQ ID NO: 7) | TAACGGCCGCGCACAAAAAAACACCCAGTC |
| | CCTGTCCAAGGGCCCGGGTGTTTTTTTGCC |
| | GATAAGTTGCTCGGCTA-RXF04176 |
| Term02857/58 | RXF2857- |
| (SEQ ID NO: 8) | TGATCAGCAAGCGCTATAAAAAATGCCCCG |
| | TATCGCAAGATACGGGGCATTTTCATTTTC |
| | AGGCCCGATAAAGCTCA-RXF02858 |
| Term04176/67- | RXF04176- |
| Term02857/58 | TAGCCGAGCAACTTATCGGCAAAAAAACAC |
| (SEQ ID NO: 9) | CCGGGCCCTTGGACAGGGACTGGGTGTTTT |
| | TTTGTGCGCGGCCGTTAACTAGTTGATCAG |
| | CAAGCGCTATAAAAAATGCCCCGTATCGCA |
| | AGATACGGGGCATTTTCATTTTCAGGCCCG |
| | ATAAAGCTCA-RXF02858 |

The Termination of COP-GFP Activity by Bidirectional Terminators

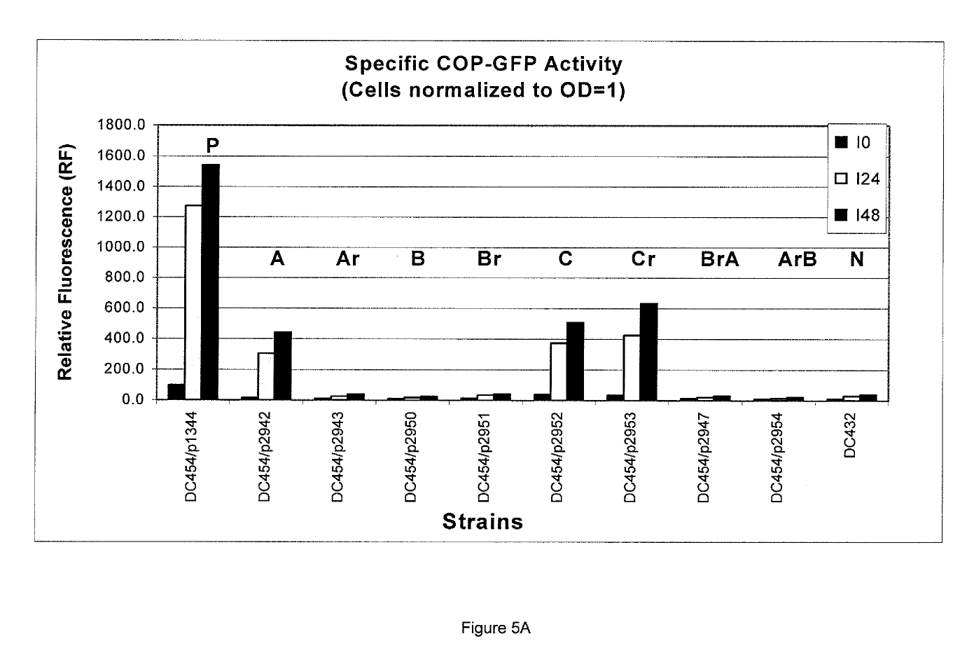

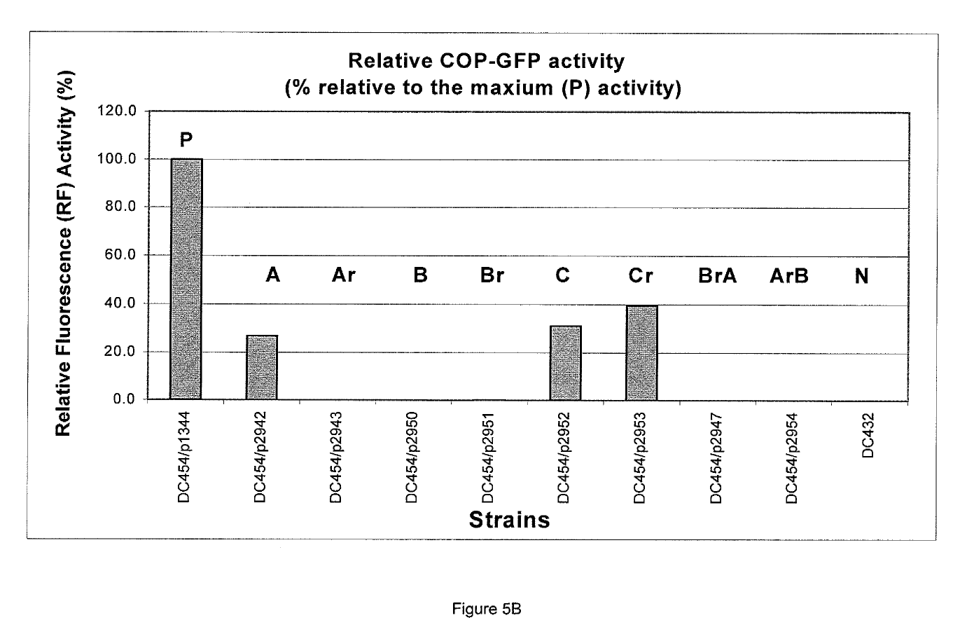

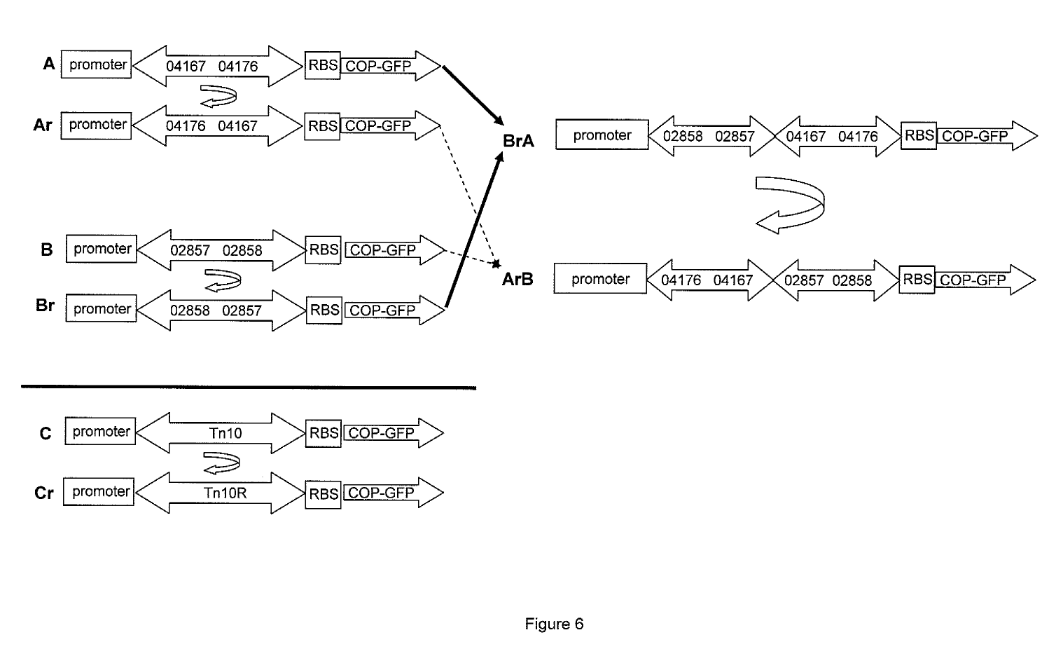

The plasmids were electroporated into P. fluorescens DC454, grown in 96-well plates and COP-GFP expression analyzed. The results are shown in FIG. 5. The results showed that insertion of the Term02857/58 (FIG. 6 constructions B and Br) as well as combined Term04167/76-Term02857/58 (FIG. 6 constructions BrA and ABr) in both orientations reduced the maximal expression of COP-GFP to less than 1%. In contrast the Term04176/67 (FIG. 6 constructions A and Ar) reduced the maximal expression to <1% in one direction (Ar) but only to ˜25% in the other (A). The bidirectional E. coli Tn10 terminator retained 30-40% of the maximal COP-GFP activity in both orientations. (FIG. 6 constructions C and Cr).

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1-32. (canceled)

33. A composition comprising a population of expression vectors, wherein members of the population of expression vectors comprise at least one type IIS restriction enzyme recognition site adjacent to a regulatory element, wherein members of the population of expression vectors comprise identical or non-identical type IIS restriction enzyme recognition sites, and wherein the regulatory element is distinct in at least two members of the population of expression vectors.

34. A method of identifying an expression construct, wherein said method comprises:
a) obtaining a population of expression vectors, wherein members of the population comprise at least one type IIS restriction enzyme recognition site adjacent to a regulatory element, wherein members of the population of expression constructs comprise identical or non-identical type IIS restriction enzyme recognition sites, and wherein the regulatory element is distinct in at least two members of the population of expression vectors;
b) cleaving the population of expression vectors obtained in step (a) with at least one type IIS restriction enzyme, wherein said at least one type IIS restriction enzyme recognizes the at least one type IIS restriction enzyme recognition site adjacent to the regulatory element, thereby producing a population of cleaved expression vectors;
c) obtaining a population of polynucleotide sequences comprising at least one coding region encoding a polypeptide of interest, wherein said population of polynucleotide sequences is ligation-compatible with said population of cleaved expression vectors; and,
d) ligating said population of polynucleotide sequences comprising at least one coding region encoding a polypeptide of interest to said population of cleaved expression vectors to produce a population of expression constructs.
e) introducing said population of expression constructs into a population of host cells to obtain a population of transformed host cells;
f) culturing the transformed host cell population under conditions that allow for the expression of a polypeptide of interest in at least one cell; and
g) identifying a host cell that expresses a sufficient level of said polypeptide of interest.

35. The composition of claim 33, wherein said at least one type IIS restriction enzyme recognition site is recognized by a type IIS restriction enzyme that cleaves DNA in a manner that leaves overhanging ends.

36. The method of claim 34, wherein said at least one type IIS restriction enzyme recognition site is recognized by a type IIS restriction enzyme that cleaves DNA in a manner that leaves overhanging ends.

37. The composition of claim 33, wherein said at least one type IIS restriction enzyme is selected from the group consisting of AarI, Acc36I, AceIII, AclWI, AcuI, AjuI, AloI, AlwI, Alw26I, AlwXI, AsuHPI, BaeI, Bbr7I, BbsI, BbvI, BbvII, Bbv16II, BccI, Bce83I, BceAI, BcefI, BciVI, BcgI, Bco5I, Bco116I, BcoKI, BfiI, BfuAI, BfuI, BinI, Bli736I, Bme585I, BmrI, BmuI, BpiI, BpmI, BpuAI, BpuEI, BpuSI, BsaI, BsaXI, Bsc91I, BscAI, Bse3DI, BseGI, BseKI, BseMI, BseMII, BseRI, BseXI, BseZI, BsgI, BslFI, BsmAI, BsmBI, BsmFI, Bso31I, BsoMAI, Bsp24I, Bsp423I, BspBS31I, BspCNI, BspIS41, BspKT51, BspLU111II, BspMI, BspPI, BspQI, BspST5I, BspTNI, BspTS5141, BsrD1, Bst6I, Bst12I, Bst19I, Bst71I, BstBS32I, BstF5I, BstFZ438I, BstGZ53I, BstH9I, BstMAI, BstOZ616I, BstV1I, BstV2I, Bst31TI, BstT35I, Bsu6I, BtgZI, BtsCI, BtsI, BveI, CjeI, CjePI, CseI, CspCI, CstMI, EacI, Eam1104I, EarI, EcuI, Eco31I, Eco57I, Eco57MI, EcoA4I, EcoO441, Esp3I, FaqI, FauI, FokI, GsuI, HgaI, Hin4I, Hin4II, HphI, HpyAV, HpyC1I, Ksp632I, LguI, LweI, MboII, MmeI, MnlI, NcuI, NmeAIII, PciSI, PhaI, PleI, PpiI, PpsI, PsrI, RleAI, SapI, SfaNI, SmuI, Sth132I, StsI, TaqII, TsoI, TspDTI, TspGWI, TstI, Tth111II, and VpaK321.

38. The method of claim 34, wherein said at least one type IIS restriction enzyme is selected from the group consisting of AarI, Acc36I, AceIII, AclWI, AcuI, AjuI, AloI, AlwI, Alw26I, AlwXI, AsuHPI, BaeI, Bbr7I, BbsI, BbvI, BbvII, Bbv16II, BccI, Bce83I, BceAI, BcefI, BciVI, BcgI, Bco5I, Bco116I, BcoKI, BfiI, BfuAI, BfuI, BinI, Bli736I, Bme585I, BmrI, BmuI, BpiI, BpmI, BpuAI, BpuEI, BpuSI, BsaI, BsaXI, Bsc91I, BscAI, Bse3DI, BseGI, BseKI, BseMI, BseMII, BseRI, BseXI, BseZI, BsgI, BslFI, BsmAI, BsmBI, BsmFI, Bso31I, BsoMAI, Bsp24I, Bsp423I, BspBS31I, BspCNI, BspIS41, BspKT51, BspLU11II, BspMI, BspPI, BspQI, BspST5I, BspTNI, BspTS5141, BsrD1, Bst6I, Bst12I, Bst19I, Bst71I, BstBS32I, BstF5I, BstFZ438I, BstGZ53I, BstH9I, BstMAI, BstOZ616I, BstV1I, BstV2I, Bst31TI, BstT35I, Bsu6I, BtgZI, BtsCI, BtsI, BveI, CjeI, CjePI, CseI, CspCI, CstMI, EacI, Eam1104I, EarI, EciI, Eco31I, Eco57I, Eco57MI, EcoA4I, EcoO441, Esp3I, FaqI, FauI, FokI, GsuI, HgaI, Hin4I, Hin4II, HphI, HpyAV, HpyC1I, Ksp632I, LguI, LweI, MboII, MmeI, MnlI, NcuI, NmeAIII, PciSI, PhaI, PleI, PpiI, PpsI, PsrI, RleAI, SapI, SfaNI, SmuI, Sth132I, StsI, TaqII, TsoI, TspDTI, TspGWI, TstI, Tth111II, and VpaK321.

39. The composition of claim 37, wherein the type IIS restriction enzyme is SapI.

40. The method of claim 38, wherein the type IIS restriction enzyme is SapI.

41. The composition of claim 33, wherein said regulatory element is selected from the group consisting of a promoter, an enhancer, an operator, a repressor, a transcription termination sequence, an untranslated region, a ribosome binding site, a translation initiation codon, a translation termination codon, a signal sequence, and a coding region encoding a peptide tag or a protease cleavage site.

42. The method of claim 34, wherein said regulatory element is selected from the group consisting of a promoter, an enhancer, an operator, a repressor, a transcription termination sequence, an untranslated region, a ribosome binding site, a translation initiation codon, a translation termination codon, a signal sequence, and a coding region encoding a peptide tag or a protease cleavage site.

43. The composition of claim 33, further comprising a population of polynucleotide sequences comprising at least one coding region encoding a polypeptide of interest.

44. The composition of claim 33, wherein members of said population of expression vectors comprise identical or non-identical polynucleotide sequences comprising at least one coding region encoding a polypeptide of interest.

45. The method of claim 34, wherein members of said population of expression vectors comprise identical or non-identical polynucleotide sequences comprising at least one coding region encoding a polypeptide of interest.

46. The composition of claim 43, wherein members of said population of polynucleotide sequences comprise a first region encoding a first polypeptide of interest, and a second region encoding a second polypeptide of interest.

47. The method of claim 45 wherein members of said population of polynucleotide sequences comprise a first region encoding a first polypeptide of interest, and a second region encoding a second polypeptide of interest

48. The composition of claim 46, wherein said first region encoding said first polypeptide of interest and said second region encoding said second polypeptide of interest are co-transcribed from a single promoter operably associated therewith, but are separately translated.

49. The composition of claim 46, wherein said first region encoding said first polypeptide of interest and said second region encoding said second polypeptide of interest are separately transcribed, each being operably associated with a separate promoter.

50. The composition of claim 49, wherein said population of polynucleotide sequences further comprises a bidirectional transcription termination sequence disposed between said region encoding said first polypeptide of interest and said region encoding said second polypeptide of interest, wherein the orientation of each polypeptide-encoding region is such that transcription of each region proceeds towards the bidirectional transcription termination sequence.

51. The composition of claim 50, wherein said population of expression vectors comprises a regulatory element operably linked to said region encoding said first polypeptide of interest and a regulatory element operably linked to said region encoding said second polypeptide of interest, wherein the orientation of each regulatory sequence is such that transcription of each region proceeds towards the bidirectional transcription termination sequence.

52. The composition of claim 50, wherein said bidirectional transcription termination sequence comprises the sequence of one of SEQ ID NOs: 7, 8 and 9.

53. The method of claim 34, wherein cleaving the population of expression vectors with said at least one type IIS restriction enzyme produces a population of cleaved expression vectors comprising at least one overhanging end.

54. The method of claim 53, wherein termini of said population of cleaved expression vectors comprise identical overhanging ends.

55. The method of claim 53, wherein termini of said population of cleaved expression vectors comprise non-identical overhanging ends.

56. The method of claim 34, wherein the host cell is a eukaryote.

57. The method of claim 34, wherein the host cell is a prokaryote.

58. The method of claim 57, wherein said prokaryote is a Pseudomonad.

59. The method of claim 58, wherein said Pseudomonad is a Pseudomonas fluorescens.

60. The method of claim 57, wherein said prokaryote is an Escherichia coli.


SEQUENCE LISTING (9 sequences)

SEQ ID NO: 1 - DNA, 30 bp, Artificial Sequence
  Oligonucleotide primer Rapid SapI minus F
atatctgcagcatacatctggaagcaaagc

SEQ ID NO: 2 - DNA, 20 bp, Artificial Sequence
  Oligonucleotide primer Rapid SapI minus R
cgcctgtacgtggccctgaa

SEQ ID NO: 3 - DNA, 125 bp, Artificial Sequence
  Oligonucleotide primer Rapid 101
atatgctcttcaggccaccgcgttggcggtcgcaacgccagcagcgacaaaagtcattgccgccatcaaacgtttcagtttcataagttacctcctactagtagattaaaattctgtttcctgtg

SEQ ID NO: 4 - DNA, 35 bp, Artificial Sequence
  Oligonucleotide primer Rapid 103
atatgctcttcataactcgagcccaaaacgaaagg

SEQ ID NO: 5 - DNA, 36 bp, Artificial Sequence
  Oligonucleotide primer JCS541
atatgctcttcagcccggacaccagaaatgcctgtt

SEQ ID NO: 6 - DNA, 44 bp, Artificial Sequence
  Oligonucleotide primer JCS542-R
atatgctcttctttactattatcaggtacctttcagccccagag

SEQ ID NO: 7 - DNA, 42 bp, Pseudomonas fluorescens
ccaagggcccgggtgtttttttgccgataagttgctcggcta

SEQ ID NO: 8 - DNA, 77 bp, Pseudomonas fluorescens
tgatcagcaagcgctataaaaaatgccccgtatcgcaagatacggggcattttcattttcaggcccgataaagctca

SEQ ID NO: 9 - DNA, 160 bp, Artificial Sequence
  Combined terminator sequence Term4167/76-Term2857/58
tagccgagcaacttatcggcaaaaaaacacccgggcccttggacagggactgggtgtttttttgtgcgcggccgttaactagttgatcagcaagcgctataaaaaatgccccgtatcgcaagatacggggcattttcattttcaggcccgataaagctca